(19) United States
(12) Patent Application Publication
(10) Pub. No.: US 2026/0207567 A1
(43) Pub. Date: Jul. 23, 2026

(54) ANTIVIRAL COMPOSITION AND A PHARMACEUTICAL COMPOSITION FOR PREVENTING OR TREATING HEPATITIS B COMPRISING SPIPERONE AS A cccDNA INHIBITOR

(71) Applicant: SEOUL NATIONAL UNIVERSITY R&DB FOUNDATION, Seoul (KR)

(72) Inventors: Bum-Joon KIM, Seoul (KR); Yu-Min CHOI, Seoul (KR); Junghwa JANG, Seoul (KR); Ziyun KIM, Seoul (KR)

(21) Appl. No.: 19/436,723

(22) Filed: Dec. 30, 2025

Related U.S. Application Data
(63) Continuation-in-part of application No. PCT/KR2025/011246, filed on Jul. 29, 2025.

(30) Foreign Application Priority Data
Aug. 1, 2024 (KR) 10-2024-0102294

(51) Int. Cl. A61K 31/438; A61K 31/675; A61K 45/06; A61P 31/20

(52) U.S. Cl. CPC A61K 31/438; A61K 31/675; A61K 45/06; A61P 31/20

(57) ABSTRACT

The present disclosure relates to: an antiviral composition comprising spiperone as a cccDNA inhibitor; and a pharmaceutical composition for the prevention or treatment of viral infection or hepatitis B, comprising spiperone as a cccDNA inhibitor. The antiviral composition comprising spiperone, of the present invention, has antiviral activity against a wide range of viruses in consideration of the cccDNA inhibitory efficacy and mechanism of action. Also, the pharmaceutical composition comprising spiperone has excellent cccDNA inhibitory efficacy, antiviral efficacy, and therapeutic efficacy against viruses, and thus can be effectively used for the prevention or treatment of viral infection such as hepatitis B. Also, spiperone has a synergistic effect with an existing antiviral agent, and thus is effective in treating viral infection.

### PRIORITY CLAIM

This application is a bypass continuation-in-part application of PCT Application No. PCT/KR2025/011246, filed Jul. 29, 2025, which claims priority to Korean Patent Application No. 10-2024-0102294, filed Aug. 1, 2024, both of which are herein incorporated by reference in their entirety.

### SEQUENCE LISTING

The instant application contains a Sequence Listing which has been submitted in ASCII format and is hereby incorporated by reference in its entirety. Said ASCII copy, created on Mar. 26, 2026, is named PX-250264-USCIP_sequence.xml and is 6453 bytes in size.

### TECHNICAL FIELD

The present disclosure relates to: an antiviral composition comprising spiperone as a cccDNA inhibitor; and a pharmaceutical composition for preventing or treating viral infection or hepatitis B, comprising spiperone as a cccDNA inhibitor.

### BACKGROUND ART

Approximately 296 million people (3.8%) worldwide are living with chronic hepatitis B, which is a disease caused by hepatitis B virus (HBV), and the number of infected people continues to increase with approximately 1.5 million new cases being infected every year. In 2019 alone, about 820,000 deaths were reported due to liver cirrhosis or liver cancer caused by HBV.

When chronic hepatitis due to HBV infection progresses, treatment is challenging and there is a high risk of developing into liver cirrhosis or liver cancer. In particular, 75% of the causative factors of liver cancer correspond to HBV infection, and the progression of chronic hepatitis increases the risk of liver cancer 20 times compared to the general population. In Korea, chronic HBV infection causes liver cancer in over 50% of cases, and liver cancer ranks as the number one cause of death among men in their 50 s, thus greatly affecting public health and being difficult to treat.

Despite the high prevalence of HBV and some effective treatments, HBV remains incurable, leaving a high unmet need for functional cure. Accordingly, the development of antiviral agents for chronic HBV virus is in great demand in the market. In particular, the HBV therapeutics market is expected to expand, and according to the recently announced “Global Hepatitis B Therapeutics Market: Growth Trends and Competitive Analysis (2022-2033)” (Report for More Insights on the Hepatitis B Tests Market Forecast 2023-2033), the global HBV therapeutics market size is expected to reach USD 10.5 billion by the end of 2029 from USD 1.6 billion in 2022, with an explosive growth of 30% per year.

When infected with HBV, cccDNA inserted into the patient's genes is difficult to remove, making a complete cure practically impossible. Therefore, for the complete cure of HBV infection, the most fundamental treatment objective currently is to either interfere with cccDNA formation or suppress the transcription thereof to completely eliminate cccDNA from a host.

Currently, any therapeutic agent from among nucleotide analogs (NAs) or PEG-IFN, which are representative therapeutic agents, has an inhibitory effect on HBV DNA, but the reduction rate of HBsAg, which is considered a major functional cure indicator associated with clinical outcomes such as liver cancer, liver dysfunction, liver transplantation, and mortality, remains inadequate at 1-2% for NAs and 4% or less for PEG-IFN, showing no significant effect in eliminating cccDNA.

Therefore, there is a growing need for effective drugs that can still provide functional treatment in the progression of chronic hepatitis.

However, new drug development requires approximately 15 years and more than one billion dollars, with substantial time needed before market launch. Most drugs fail to receive approval in Phase 2 clinical trials, with at least 50% due to insufficient efficacy and 25% primarily caused by toxic reactions. Consequently, the proportion of drugs that pass from the drug target stage to final approval is only 1-3%.

Drug repositioning is a development strategy that involves repurposing drugs that were developed and approved for different uses, or drugs currently in clinical trials but not yet approved, by switching their intended application. This strategy uses drugs that have already demonstrated some level of effect and safety, merely changing the application thereof. Drug repositioning has become a critical strategy since the COVID-19 pandemic, when the urgent need for therapeutic agents and vaccines emerged, and drug repositioning is actively being developed within the same therapeutic area and across different therapeutic areas.

Such drug repositioning offers clear advantages, including reducing overall development costs, reducing development risks due to substances with proven safety, and substantially shortening development timelines because safety evaluations and drug formulations are already established.

Therefore, the inventors of the present application aimed to develop uses of existing drugs as novel antiviral agents and for the treatment of viral infection, through drug repositioning. Through screening and mechanistic studies, the inventors of the present application derived use of spiperone as a novel antiviral agent and for the treatment of viral infection.

Spiperone is a dopamine antagonist that binds to dopamine and serotonin receptors, which was approved in Japan for the treatment of schizophrenia.

The inventors of the present application had conducted extensive research and as a result, discovered the mechanism by which spiperone increases antiviral activity in the body through intrinsic activation of type I interferon via the PERK-elF2a-ATF4-IFN pathway, thereby deriving use of spiperone for the treatment of viral infection and as a novel antiviral agent against various viruses as well as DNA viruses that produce cccDNA in host cells, such as HBV.

In addition, the inventors of the present application derived novel use of spiperone, which is innovative and has increased safety to promote HBV cure, for the treatment of HBV infection by overcoming the limitations of existing HBV therapeutic agents through spiperone, which has a mechanism different from various known antiviral agents, and by eliminating cccDNA, which is a therapeutic challenge in HBV infection.

### DISCLOSURE OF INVENTION

### Technical Problem

An aspect of the present disclosure is to provide an antiviral composition comprising spiperone.

Another aspect is to provide antiviral use of spiperone.

Another aspect is to provide use of spiperone for the preparation of an antiviral composition.

Another aspect is to provide a pharmaceutical composition for the prevention or treatment of viral infection, comprising spiperone or a pharmaceutically acceptable salt thereof as an active ingredient.

Another aspect is to provide a method for inhibiting or removing a viral cccDNA expression in a nucleus of a host cell, comprising administering spiperone or a pharmaceutically acceptable salt thereof to a subject in need thereof.

Another aspect is to provide a method for inhibiting or removing a virus that produces cccDNA in a nucleus of a host cell, comprising administering spiperone or a pharmaceutically acceptable salt thereof to a subject in need thereof.

Another aspect is to provide a method of preventing or treating virus infection, comprising administering spiperone or a pharmaceutically acceptable salt thereof to a subject in need thereof.

Another aspect is to provide use of spiperone or a pharmaceutically acceptable salt thereof for the preparation of a pharmaceutical composition for the prevention or treatment of viral infection.

Another aspect is to provide use of spiperone or a pharmaceutically acceptable salt thereof for the prevention or treatment of viral infection.

Another aspect is to provide a pharmaceutical composition for the prevention or treatment of hepatitis B, comprising spiperone or a pharmaceutically acceptable salt thereof as an active ingredient.

Another aspect is to provide a method of preventing or treating hepatitis B, comprising administering spiperone or a pharmaceutically acceptable salt thereof to a subject in need thereof.

Another aspect is to provide use of spiperone or a pharmaceutically acceptable salt thereof for the preparation of a pharmaceutical composition for the prevention or treatment of hepatitis B.

Another aspect is to provide use of spiperone or a pharmaceutically acceptable salt thereof for the prevention or treatment of hepatitis B.

Another aspect is to provide an antiviral health functional food comprising spiperone.

Another aspect is to provide use of spiperone for the preparation of an antiviral health functional food.

Another aspect is to provide an antiviral feed composition comprising spiperone.

Another aspect is to provide use of spiperone for the preparation of an antiviral feed composition.

### Solution to Problem

An aspect of the present disclosure provides an antiviral composition comprising spiperone.

Another aspect provides antiviral use of spiperone.

Another aspect provides use of spiperone for the preparation of an antiviral composition.

Spiperone is a dopamine antagonist that binds to dopamine and serotonin receptors.

The spiperone has CAS No. 749-02-0 and has a structure of a compound of Formula 1:

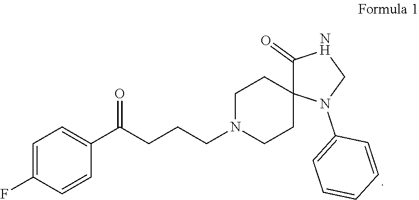 [SMILES: O=C(CCCN1CCC2(CC1)C(=O)NCN2c1ccccc1)c1ccc(F)cc1]

The term “antiviral” as used herein refers to the ability to resist viruses, encompassing resistance to viruses or suppression of viral reproduction, or all mechanisms that defend against the actions of viruses.

In an embodiment, the antiviral composition may have antiviral activity against viruses, such as inhibiting viral gene expression, inhibiting protein expression, or suppressing viral replication.

In an embodiment, the virus may be one or more selected from the group consisting of hepatitis B virus, adenovirus, herpes virus, herpes simplex virus 1 (HSV-1), herpes simplex virus 2 (HSV-2), varicella-zoster virus (VZV), Epstein-Barr virus (EBV), cytomegalovirus (CMV), human herpesvirus 6 (HHV-6), human herpesvirus 7 (HHV-7), Kaposi's sarcoma-associated herpesvirus (KSHV/HHV-8), variola virus, Monkeypox virus, human papillomavirus (HPV), JC virus, BK virus, Merkel cell polyomavirus (MCV), human parvovirus B19, Bocavirus, hepatitis D virus, coronavirus, Severe Acute Respiratory Syndrome Coronavirus (SARS-COV), hepatitis A virus, hepatitis C virus, COVID-19 or Severe Acute Respiratory Syndrome Coronavirus-2 (SARS-COV-2), Human Immunodeficiency Virus (HIV), severe fever with thrombocytopenia syndrome virus, poliovirus, dengue virus, measles virus, influenza virus, rotavirus, herpes virus, flavivirus, togavirus, rubivirus, pestivirus, Marburg virus, encephalitis virus, Japanese encephalitis virus, Ebola virus, Nipah virus, and rhinovirus.

In an embodiment, the virus may be one or more selected from the group consisting of DNA viruses, RNA viruses, and retroviruses. Specifically, the virus may be a DNA virus.

In an embodiment, the virus may be a virus that produces cccDNA within the nucleus of a host cell.

In an embodiment, the antiviral composition may have cccDNA inhibitory activity and/or removal activity.

Covalently closed circular DNA (cccDNA) is a circular, closed-form DNA molecule that is produced within the nucleus of host cells during the replication of certain viruses, and plays a critical role in viral replication and maintaining persistent infection. Therefore, inhibiting cccDNA is a major antiviral strategy.

In an embodiment, the antiviral composition may have antiviral activity through a mechanism that induces endoplasmic reticulum stress (ER stress) and interferon. Specifically, the antiviral composition may have antiviral activity by activating the PERK-elF2α-ATF4 signaling pathway, which is one of the ER stress pathways, and inducing the secretion of type I interferon.

Specifically, the composition comprising spiperone may exhibit antiviral activity by inducing the ER stress pathway centered on the PERK-elF2α-ATF4 axis, thereby activating the STING-TBK1-IRF3/7 signaling pathway via the leakage of viral DNA in the cytoplasm and the activation of IFI16, which is a DNA sensor, and accordingly inducing the expression of type I interferon (type I IFN) and interferon-stimulated genes (ISGs).

More specifically, spiperone may significantly activate ER stress responses by inducing phosphorylation of PERK, phosphorylation of elF2α, and expression of downstream transcription factor ATF4 in cells, which may lead to increased mtROS generation, leakage of damaged mtDNA into the cytoplasm, and accumulation of oxidized DNA, thereby inducing recognition by IFI16 and activation of the type I IFN signaling pathway.

In an embodiment, the antiviral composition may further include an antiviral agent.

The antiviral agent may be one or more selected from the group consisting of tenofovir, entecavir, adefovir, sofosbuvir, cidofovir, acyclovir, famciclovir, valacyclovir, ganciclovir, amprenavir, abacavir, ansamycin, darunavir, delavirdine, efavirenz, etravirine, hypericin, indinavir, lamivudine, lobucavir, nelfinavir, nevirapine, novapren, ritonavir, saquinavir, stavudine, tipranavir, virazole, ribavirin, zalcitabine, zidovudine, maraviroc, raltegravir, elvitegravir, didanosine, emtricitabine, lopinavir, atazanavir, enfuvirtide, and clevudine.

Another aspect provides a pharmaceutical composition for the prevention or treatment of viral infection, comprising spiperone or a pharmaceutically acceptable salt thereof as an active ingredient.

Another aspect provides a method of preventing or treating viral infection, the method comprising administering spiperone or a pharmaceutically acceptable salt thereof to a subject in need thereof.

Another aspect provides use of spiperone or a pharmaceutically acceptable salt thereof for the preparation of a pharmaceutical composition for the prevention or treatment of viral infection.

Another aspect provides use of spiperone or a pharmaceutically acceptable salt thereof for the prevention or treatment of viral infection.

Another aspect provides a method of preventing or treating viral infection, the method comprising administering spiperone to a subject in need thereof.

In the pharmaceutical composition for the prevention or treatment of viral infection or the method of preventing or treating viral infection, according to an embodiment, the virus may be one or more selected from the group consisting of hepatitis B virus, adenovirus, herpes virus, herpes simplex virus 1 (HSV-1), herpes simplex virus 2 (HSV-2), varicella-zoster virus (VZV), Epstein-Barr virus (EBV), cytomegalovirus (CMV), human herpesvirus 6 (HHV-6), human herpesvirus 7 (HHV-7), Kaposi's sarcoma-associated herpesvirus (KSHV/HHV-8), variola virus, Monkeypox virus, human papillomavirus (HPV), JC virus, BK virus, Merkel cell polyomavirus (MCV), human parvovirus B19, Bocavirus, hepatitis D virus, coronavirus, Severe Acute Respiratory Syndrome Coronavirus (SARS-CoV), hepatitis A virus, hepatitis C virus, COVID-19 or Severe Acute Respiratory Syndrome Coronavirus-2 (SARS-COV-2), Human Immunodeficiency Virus (HIV), severe fever with thrombocytopenia syndrome virus, poliovirus, dengue virus, measles virus, influenza virus, rotavirus, herpes virus, flavivirus, togavirus, rubivirus, pestivirus, Marburg virus, encephalitis virus, Japanese encephalitis virus, Ebola virus, Nipah virus, and rhinovirus.

In the pharmaceutical composition for the prevention or treatment of viral infection or the method of preventing or treating viral infection, according to an embodiment, the virus may be one or more selected from the group consisting of DNA viruses, RNA viruses, and retroviruses. Specifically, the virus may be a DNA virus.

In the pharmaceutical composition for the prevention or treatment of viral infection or the method of preventing or treating viral infection, according to an embodiment, the virus may be a virus that produces cccDNA in the nucleus of a host cell.

In the pharmaceutical composition for the prevention or treatment of viral infection or the method of preventing or treating viral infection, according to an embodiment, the pharmaceutical composition or the treatment method may have cccDNA inhibitory activity and/or removal activity.

Covalently closed circular DNA (cccDNA) is a circular, closed-form DNA molecule that is produced within the nucleus of host cells during the replication of certain viruses, and plays a critical role in viral replication and maintaining persistent infection. Therefore, inhibiting cccDNA is a major strategy for the prevention or treatment of viral infection.

In the pharmaceutical composition for the prevention or treatment of viral infection or the method of preventing or treating viral infection, according to an embodiment, the pharmaceutical composition or the treatment method may have preventive or therapeutic activity for viral infection through a mechanism that induces endoplasmic reticulum stress (ER stress) and interferon. Specifically, the pharmaceutical composition or the treatment method may have preventive or therapeutic activity for viral infection by activating the PERK-elF2α-ATF4 signaling pathway, which is one of the ER stress pathways, and inducing the secretion of type I interferon.

Specifically, the composition comprising spiperone may exhibit preventive or therapeutic activity for viral infection by inducing the ER stress pathway centered on the PERK-elF2α-ATF4 axis, thereby activating the STING-TBK1-IRF3/7 signaling pathway via the leakage of viral DNA in the cytoplasm and the activation of IFI16, which is a DNA sensor, and accordingly inducing the expression of type I interferon (type I IFN) and interferon-stimulated genes (ISGs).

More specifically, spiperone may significantly activate ER stress responses by inducing phosphorylation of PERK, phosphorylation of elF2α, and expression of downstream transcription factor ATF4 in cells, which may lead to increased mtROS generation, leakage of damaged mtDNA into the cytoplasm, and accumulation of oxidized DNA, thereby inducing recognition by IFI16 and activation of the type I IFN signaling pathway.

The term “viral infection” or “virus infection” as used herein refers to a disease caused by a virus, encompassing a comprehensive concept that includes various symptoms and conditions resulting from viral infection. “Viral infection” includes “viral infectious disease.”

In an embodiment, the pharmaceutical composition for the prevention or treatment of viral infection may further include an antiviral agent.

In an embodiment, the method of preventing or treating viral infection may further include administering an antiviral agent.

The antiviral agent may be one or more selected from the group consisting of tenofovir, entecavir, adefovir, sofosbuvir, cidofovir, acyclovir, famciclovir, valacyclovir, ganciclovir, amprenavir, abacavir, ansamycin, darunavir, delavirdine, efavirenz, etravirine, hypericin, indinavir, lamivudine, lobucavir, nelfinavir, nevirapine, novapren, ritonavir, saquinavir, stavudine, tipranavir, virazole, ribavirin, zalcitabine, zidovudine, maraviroc, raltegravir, elvitegravir, didanosine, emtricitabine, lopinavir, atazanavir, enfuvirtide, and clevudine.

Another aspect provides a pharmaceutical composition for the prevention or treatment of hepatitis B, comprising spiperone or a pharmaceutically acceptable salt thereof as an active ingredient.

Another aspect provides a method of preventing or treating hepatitis B, comprising administering spiperone or a pharmaceutically acceptable salt thereof to a subject in need thereof.

Another aspect provides use of spiperone or a pharmaceutically acceptable salt thereof for the preparation of a pharmaceutical composition for the prevention or treatment of hepatitis B.

Another aspect provides use of spiperone or a pharmaceutically acceptable salt thereof for the prevention or treatment of hepatitis B.

Another aspect provides a method of preventing or treating hepatitis B, the method comprising administering spiperone to a subject in need thereof.

In the pharmaceutical composition for the prevention or treatment of hepatitis B or the method of preventing or treating hepatitis B, according to an embodiment, the pharmaceutical composition or the treatment method may have cccDNA inhibitory activity and/or removal activity.

Covalently closed circular DNA (cccDNA) is a circular, closed-form DNA molecule that is produced within the nucleus of host cells during the replication of certain viruses, and plays a critical role in viral replication and maintaining persistent infection. In particular, cccDNA is a major factor that causes chronic infection with hepatitis B virus (HBV). Currently, no drug has been developed that is effective in inhibiting cccDNA of HBV, and inhibiting cccDNA is a major strategy for the prevention or treatment of hepatitis B.

In the pharmaceutical composition for the prevention or treatment of hepatitis B or the method of preventing or treating hepatitis B, according to an embodiment, the pharmaceutical composition or the treatment method may have preventive or therapeutic activity for viral infection through a mechanism that induces endoplasmic reticulum stress (ER stress) and interferon. Specifically, the pharmaceutical composition or the treatment method may have preventive or therapeutic activity for hepatitis B by activating the PERK-elF2α-ATF4 signaling pathway, which is one of the ER stress pathways, and inducing the secretion of type I interferon. Specifically, the composition comprising spiperone may exhibit preventive or therapeutic activity for hepatitis B by inducing an ER stress pathway centered on the PERK-elF2α-ATF4 axis, thereby activating the STING-TBK1-IRF3/7 signaling pathway through the leakage of HBV DNA into the cytoplasm and the activation of IFI16, which is a DNA sensor, thereby inducing the expression of type I interferon (type I IFN) and interferon-stimulated genes (ISGs).

More specifically, spiperone may significantly activate ER stress responses by inducing phosphorylation of PERK, phosphorylation of elF2α, and expression of downstream transcription factor ATF4 in cells, which may lead to increased mtROS generation, leakage of damaged mtDNA into the cytoplasm, and accumulation of oxidized DNA, thereby inducing recognition by IFI16 and activation of the type I IFN signaling pathway.

In an embodiment, the pharmaceutical composition for the prevention or treatment of hepatitis B may further include an antiviral agent.

In an embodiment, the method of preventing or treating hepatitis B may further include administering an antiviral agent.

The antiviral agent may be one or more selected from the group consisting of tenofovir, entecavir, adefovir, sofosbuvir, cidofovir, acyclovir, famciclovir, valacyclovir, ganciclovir, amprenavir, abacavir, ansamycin, darunavir, delavirdine, efavirenz, etravirine, hypericin, indinavir, lamivudine, lobucavir, nelfinavir, nevirapine, novapren, ritonavir, saquinavir, stavudine, tipranavir, virazole, ribavirin, zalcitabine, zidovudine, maraviroc, raltegravir, elvitegravir, didanosine, emtricitabine, lopinavir, atazanavir, enfuvirtide, and clevudine.

The term “comprising as an active ingredient” as used herein means that the pharmaceutical composition includes an effective amount sufficient to exhibit a preventive or therapeutic effect for viral infection or hepatitis B.

The term “pharmaceutically acceptable” as used herein means a substance that can be effectively used for the intended purpose within the scope of medical and pharmaceutical judgement, without causing excessive toxicity, irritation, allergic reactions, or the like.

The term “pharmaceutically acceptable salt” as used herein means a salt according to an aspect of the present disclosure that is pharmaceutically acceptable and has the desirable pharmacological activity of a parent compound. Salts of the parent compound may be synthesized from the parent compound containing a basic or acidic moiety by conventional chemical methods. In general, these salts may be prepared by reacting the free acid form of these compounds with a stoichiometric amount of an appropriate base, such as sodium, calcium, magnesium, or potassium, or by reacting the free base form of these compounds with a stoichiometric amount of an appropriate acid. These reactions are typically performed in water, an organic solvent, or a mixture thereof. In general, when feasible, a non-aqueous medium such as ether, ethyl acetate, ethanol, isopropanol, or acetonitrile may be used. The pharmaceutically acceptable salts include both addition salts of acids or bases and stereochemically isomeric forms thereof, and may be, for example, addition salts of organic or inorganic acids. These salts include any salt that maintains the activity of the parent compound and does not cause undesirable effects in a subject, and are not particularly limited.

These salts include inorganic and organic salts, and the inorganic and organic salts may be, for example, acetic acid, nitric acid, aspartic acid, sulfonic acid, sulfuric acid, maleic acid, glutamic acid, formic acid, succinic acid, phosphoric acid, phthalic acid, tannic acid, tartaric acid, hydrobromic acid, propionic acid, benzenesulfonic acid, benzoic acid, stearic acid, lactic acid, bicarbonic acid, bisulfuric acid, bitartaric acid, oxalic acid, butyric acid, calcium edetate, carbonic acid, chlorobenzoic acid, citric acid, edetate acid, toluenesulfonic acid, fumaric acid, gluceptic acid, oxycinnamic acid, pamoic acid, gluconic acid, methylnitric acid, malonic acid, hydrochloric acid, hydroiodic acid, hydroxynaphthoic acid, isethionic acid, lactobionic acid, mandelic acid, mucic acid, naphsilic acid, muconic acid, p-nitromethanesulfonic acid, hexamic acid, pantothenic acid, monohydrogen phosphate, dihydrogen phosphate, salicylic acid, sulfamic acid, sulfanilic acid, and methanesulfonic acid. In other embodiments, the salt forms include salts of alkali and alkaline earth metals, such as ammonium salts, lithium salts, sodium salts, potassium salts, magnesium salts, and calcium salts, salts with organic bases, for example, benzathine, N-methyl-D-glucamine, and hydrabamine salts, and salts with amino acids such as arginine and lysine. In other embodiments, these salt forms may be converted to free forms by treatment with a suitable base or acid.

The term “prevention” as used herein means all actions that inhibit or delay the onset of a target disease, the term “treatment” as used herein means all actions that alleviate or beneficially change symptoms of a target disease and associated metabolic abnormalities via administration of the pharmaceutical composition according to the present disclosure, and the term “amelioration” as used herein means all actions that reduce parameters related to a target disease, for example, the severity of symptoms, via administration of a composition according to the present disclosure.

In an embodiment, the pharmaceutical composition for the prevention or treatment of viral infection or the pharmaceutical composition for the prevention or treatment of hepatitis B may be co-administered with an antiviral agent. The antiviral agent is as described above.

The term “co-administration” as used herein may be achieved by administering individual ingredients of therapy simultaneously, sequentially, in reverse order, or individually. A combination therapy effect is obtained by a method such as administering two or more drugs simultaneously, sequentially, or in reverse order, or alternately administering two or more drugs at regular or unspecified intervals, and combination therapy may be defined as, but is not limited to, providing a synergistic effect while efficacy measured through response level, response rate, time to disease progression, or survival period, is therapeutically superior to efficacy that can be obtained by administering one or the remaining ingredients of the combination therapy at conventional doses. Co-administration includes simultaneous administration or sequential administration in any order.

In this specification, the combination therapy of spiperone and an antiviral agent according to the present invention may provide a “synergistic effect,” i.e., indicating that an effect achieved in case that active substances are used together is greater than the sum of effects achieved in case that the active substances are used individually. The synergistic effect may be achieved in case that active substances are: (1) co-formulated and combined in a unit dosage form and administered or delivered simultaneously; (2) delivered sequentially, alternately, or in parallel as separate formulations; or (3) administered by some other methods. In the case of alternation therapy, the synergistic effect may be achieved, for example, by sequential administration or delivery of active substances via individual injection using individual syringes. A “synergistic combination” produces an effect that is superior to the combined effects of individual active substances in a combination.

The combination therapy may provide an “additive effect,” i.e., indicating that an effect achieved in case that active substances are used together is equivalent to the combined effects of the individual active substances.

In this specification, the terms “subject” and “patient” are used interchangeably. The subject may be an animal. In some embodiments, the subject is a mammal, such as a non-human animal (e.g., a cow, a pig, a horse, a cat, a dog, a rat, a mouse, a monkey, or other primates). In some embodiments, the subject is a cynomolgus monkey. In some embodiments, the subject is a human.

The term “therapeutically effective amount” as used herein means the amount of a drug, e.g., spiperone, that is effective in achieving the desired therapeutic or prophylactic result. In some embodiments, the desired result is the treatment of a disease or disorder in a subject. A therapeutically effective dosage level may be determined according to factors comprising the type of disease of a patient, the severity of disease, the activity of drugs, sensitivity to drugs, administration time, administration route, excretion route, treatment period, and simultaneously used drugs, and factors well known in the medical field. The composition of the present disclosure may be administered as an individual therapeutic agent or in combination with other therapeutic agents, and may be administered sequentially or simultaneously with conventional therapeutic agents, and may be administered in a single dose or multiple doses. In other words, the total effective amount of the composition of this specification may be administered to a patient as a single dose, or may be administered by a fractionated treatment protocol in which the composition is administered in multiple doses over an extended period. It is important to administer the composition in the minimum amount that enables achievement of the maximum effects without side effects in consideration of all the above-described factors, and this may be easily determined by those of ordinary skill in the art.

In this specification, terms such as “treating” or “treatment” or “for treating” or “amelioration” or “for ameliorating” refer to therapeutic measures that cure, delay, alleviate symptoms of, and/or halt the progression of a diagnosed pathological condition or disorder. Therefore, the subject in need of treatment includes those who have been diagnosed with a disorder or those in whom a disorder is suspected.

In some embodiments, the pharmaceutical composition for the prevention or treatment of viral infection or the pharmaceutical composition for the prevention or treatment of hepatitis B, of this specification, may further include a pharmaceutically acceptable carrier and may be formulated together with the carrier.

The term “pharmaceutically acceptable carrier” as used herein refers to a carrier or diluent that does not stimulate the organism and does not inhibit the biological activity and properties of the administered compound. For compositions formulated as liquid solutions, pharmaceutically acceptable carriers include sterile and biocompatible substances such as saline, sterile water, Ringer's solution, buffered saline, albumin injection solution, dextrose solution, maltodextrin solution, glycerol, ethanol, and mixtures of one or more of these components, and may additionally include other conventional additives such as antioxidants, buffers, and preservatives as needed. In other embodiments, diluents, dispersants, surfactants, binders, and lubricants may further be added to formulate the composition into: injectable formulations such as aqueous solutions, suspensions, and emulsions; pills; capsules; granules; or tablets.

The pharmaceutical composition and the composition comprising a pharmaceutically acceptable carrier, according to an embodiment of this specification, may be applied to any formulation comprising the pharmaceutical composition as an active ingredient, may be prepared as an oral or parenteral formulation, and may be formulated in a unit dosage form for ease of administration and uniformity of dosage. The pharmaceutical formulations of this specification include forms suitable for oral, rectal, nasal, topical (comprising buccal and sublingual), subcutaneous, vaginal, or parenteral (comprising intramuscular, subcutaneous, and intravenous) administration, or forms suitable for administration by inhalation or insufflation.

Oral formulations including the composition of this specification as an active ingredient may be formulated, for example, as tablets, troches, lozenges, water-soluble or oil-based suspensions, prepared powders or granules, emulsions, hard or soft capsules, syrups, or elixirs.

Parenteral formulations including the composition of this specification as an active ingredient may be formulated as injectable forms such as subcutaneous, intravenous, or intramuscular injections, suppository insertions, or spray formulations such as aerosols enabling inhalation through the respiratory tract. To formulate into an injectable formulation, the composition of this specification may be mixed with a stabilizer or buffer in water to prepare a solution or suspension, which may then be formulated into unit dosage forms in ampoules or vials.

The dosage of the pharmaceutical composition of this specification varies depending on the patient's body weight, age, gender, health condition, diet, administration time, administration method, excretion rate, and the severity of disease. Upon parenteral administration, a daily dosage ranges preferably from 0.01 μg to 100 mg per kg of body weight per day, more preferably from 1 μg to 50 mg. However, the dosage may increase or decrease depending on administration route, the severity of obesity, gender, body weight, age, and the like, and thus the dosage should not be construed as limiting the scope of this specification in any way.

Another aspect provides an antiviral health functional food including spiperone.

Another aspect provides use of spiperone for the preparation of an antiviral health functional food.

In the antiviral health functional food according to an embodiment of the present disclosure, “virus,” “antiviral,” and “antiviral activity” are as described above in relation to the antiviral composition of this specification.

Another aspect provides a health functional food for the prevention or amelioration of viral infection, including spiperone.

Another aspect provides use of spiperone for the preparation of a health functional food for the prevention or amelioration of viral infection.

Another aspect provides a health functional food for the prevention or amelioration of hepatitis B, including spiperone.

Another aspect provides use of spiperone for the preparation of a health functional food for the prevention or amelioration of hepatitis B.

In the health functional food for the prevention or amelioration of viral infection or the health functional food for the prevention or amelioration of hepatitis B, according to an embodiment of the present disclosure, “virus,” “viral infection,” “prevention,” “amelioration,” “preventive or therapeutic activity for viral infection,” and “preventive or therapeutic activity for hepatitis B” are as described above in relation to the antiviral composition of this specification.

In an embodiment, the health functional food may further include an antiviral agent, and the antiviral agent is as described above.

In an embodiment, the health functional food may further include a sitologically acceptable salt (e.g., a sitologically acceptable salt of spiperone).

The term “sitologically acceptable salt” as used herein refers to a formulation of the compound that does not cause severe irritation to an organism to which the compound is administered and does not impair the biological activity and physical properties of the compound. For example, the sitologically acceptable salt may be obtained by reacting the compound with: an inorganic acid such as hydrochloric acid, hydrobromic acid, sulfuric acid, nitric acid, phosphoric acid, or the like; sulfonic acid such as methanesulfonic acid, ethanesulfonic acid, p-toluenesulfonic acid, or the like; or an organic carboxylic acid such as tartaric acid, formic acid, citric acid, acetic acid, trichloroacetic acid, trifluoroacetic acid, capric acid, isobutanoic acid, malonic acid, succinic acid, phthalic acid, gluconic acid, benzoic acid, lactic acid, fumaric acid, maleic acid, salicylic acid, or the like. In other embodiments, the sitologically acceptable salt may be obtained by reacting the compound with a base to form: salts such as ammonium salts, alkali metal salts such as sodium or potassium salts, alkaline earth metal salts such as calcium or magnesium salts, and the like; salts of organic bases such as dicyclohexylamine, N-methyl-D-glucamine, tris(hydroxymethyl)methylamine, and the like; and salts of amino acids such as arginine, lysine, and the like, but the present disclosure is not limited thereto.

In this specification, the health functional food may be formulated into any one form selected from the group consisting of powder, tablets, capsules, pills, granules, and liquids, according to conventional methods known in the art, but the present disclosure is not limited thereto. The health functional food may be manufactured in various forms using methods known in the art.

In other embodiments, the health functional food may be prepared in the form of a composition by mixing with substances or active ingredients known to have antiviral activity.

In other embodiments, the health functional food of the present disclosure may include conventional food additives, and suitability as the “food additives” shall be determined by the specifications and standards for the relevant item in accordance with the general rules and standard test methods of the Food Additive Standards approved by the Ministry of Food and Drug Safety, unless otherwise specified. The items listed in the “Food Additive Standards” may include, for example: chemical synthetic compounds such as ketones, glycine, potassium citrate, nicotinic acid, cinnamic acid, and the like; natural additives such as persimmon pigment, licorice extract, crystalline cellulose, kaoliang pigments, guar gum, and the like; and mixed preparations such as sodium L-glutamate preparations, alkaline agents for noodles, preservative preparations, tar color preparations, and the like.

In addition to the additives, the health functional food of the present disclosure may include various nutrients, vitamins, electrolytes, flavoring agents, coloring agents, pectic acid and salts thereof, alginic acid and salts thereof, organic acids, protective colloidal thickeners, pH regulators, stabilizers, preservatives, glycerin, alcohol, carbonating agents used in carbonated beverages, and the like. In other embodiments, the composition of the present disclosure may include fruit pulp for producing natural fruit juice, fruit juice beverages, and vegetable beverages. These ingredients may be used alone or in combination.

Another aspect provides an antiviral feed composition comprising spiperone.

Another aspect provides use of spiperone for the preparation of an antiviral feed composition.

The antiviral feed composition of the present disclosure according to an embodiment of the present disclosure has antiviral activity, and in the antiviral feed composition of the present disclosure, “virus,” “antiviral,” and “antiviral activity” are as described above in relation to the antiviral composition of this specification.

Another aspect provides a feed composition for the prevention or amelioration of viral infection, comprising spiperone.

Another aspect provides use of spiperone for the preparation of a feed composition for the prevention or amelioration of viral infection.

Another aspect provides a feed composition for the prevention or amelioration of hepatitis B, comprising spiperone.

Another aspect provides use of spiperone for the preparation of a feed composition for the prevention or amelioration of hepatitis B.

In the feed composition for the prevention or amelioration of viral infection or the feed composition for the prevention or amelioration of hepatitis B, according to an embodiment of the present disclosure, “virus,” “viral infection,” “prevention,” “amelioration,” “preventive or therapeutic activity for viral infection,” and “preventive or therapeutic activity for hepatitis B” are as described above in relation to the antiviral composition of this specification.

In an embodiment, the feed composition may further include an antiviral agent, and the antiviral agent is as described above.

The term “feed composition” as used herein may refer to any natural or artificial dietary regimen, single meal, or components of such a single meal, particularly those intended for animals to eat, consume, digest, or suitable for such purposes.

The feed composition may include nutrients such as energy, proteins, lipids, vitamins, minerals, and the like, required by the subject consuming the feed, but the present disclosure is not particularly limited thereto. The subject refers to a subject being raised and includes, without limitation, any living organism capable of consuming the feed of the present disclosure, including pets and livestock.

The type of feed is not particularly limited, and feed commonly used in the relevant technical field may be used. Non-limiting examples of the feed include: plant-based feeds such as cereals, root crops, food processing by-products, algae, fibrous materials, pharmaceutical by-products, oils and fats, starches, oil cakes, grain by-products, and the like; and animal-based feeds such as proteins, inorganic substances, oils and fats, minerals, oils and fats, single-cell proteins, zooplankton, foodstuffs, or the like. These may be used alone or a mixture of two or more of these materials may be used.

The feed composition according to the present disclosure may be prepared by adding spiperone of the present disclosure within an appropriate effective concentration range in accordance with various feed manufacturing methods known in the art.

The feed composition according to the present disclosure may be applied without limitation to any subject for which the antiviral effect according to the present disclosure is targeted. For example, the feed composition may also be applied to any subject, including: non-human animals such as monkeys, dogs, cats, rabbits, guinea pigs, rats, mice, cows, sheep, pigs, goats, and the like; birds; fish; and the like.

### Advantageous Effects of Invention

An antiviral composition comprising spiperone of the present disclosure has antiviral activity against a wide range of viruses in consideration of the cccDNA inhibitory efficacy and mechanism of action. Also, a pharmaceutical composition comprising spiperone has excellent cccDNA inhibitory efficacy, antiviral efficacy and therapeutic efficacy against viruses, and thus can be effectively used for the prevention or treatment of viral infection such as hepatitis B. Also, spiperone exhibits a synergistic effect with existing antiviral agents, and thus is effective in the treatment of viral infection.

### BRIEF DESCRIPTION OF DRAWINGS

FIG. 1 is a schematic view for evaluating the antiviral activity of spiperone (in vitro).

FIG. 2 is a graph for evaluating the cytotoxicity of spiperone.

FIG. 3 is a graph showing the results of evaluating the levels of HBsAg, HBeAg, HBV DNA, and pgRNA upon treatment with spiperone in an in vitro system.

FIG. 4 illustrates the results of measuring the levels of Capsid and GAPDH upon treatment with spiperone in an in vitro system by Western blot analysis.

FIG. 5 illustrates the results of measuring the levels of HBcAg and Capsid upon treatment with spiperone in an in vitro system by Confocal analysis.

FIG. 6 illustrates the results of measuring cccDNA levels upon treatment with spiperone in an in vitro system by agarose gel electrophoresis analysis.

FIG. 7 illustrates the results of measuring cccDNA levels upon treatment with spiperone in an in vitro system by RT-qPCR analysis.

FIG. 8 illustrates Southern Blot images showing cccDNA band in HepG2.2.15 and HepG2-NTCP-C4 cells treated with PBS or Spiperone.

FIG. 9 illustrates the results of measuring HBsAg OD400 levels and cccDNA levels upon treatment with spiperone in an in vitro system by ELISA and qPCR analysis.

FIG. 10 illustrates the results of measuring cccDNA levels upon treatment with spiperone in an in vitro system by agarose gel electrophoresis analysis.

FIG. 11 illustrates the results of HBV antiviral efficacy using HepaRG.

FIG. 12 and FIG. 13 illustrate the results of evaluating the synergistic effect of spiperone and tenofovir.

FIG. 14 is a schematic view for evaluating the antiviral activity of spiperone (in vivo).

FIG. 15 illustrates an in vivo experimental schedule for evaluating the antiviral activity and viral infection treatment efficacy of spiperone upon intraperitoneal injection.

FIG. 16 is a graph showing the results of evaluating the levels of HBsAg, HBeAg, HBV DNA, and pgRNA upon treatment with spiperone (intraperitoneal injection) in an in vivo system.

FIG. 17 illustrates the results of immunohistochemical (IHC) staining analysis for HBcAg in liver tissue upon treatment with spiperone (intraperitoneal injection) in an in vivo system.

FIG. 18 illustrates the results of fluorescent immunostaining analysis for the nucleus and HBsAg in liver tissue upon treatment with spiperone (intraperitoneal injection) in an in vivo system.

FIG. 19 illustrates an in vivo experimental schedule for evaluating the antiviral activity and viral infection treatment efficacy of spiperone upon oral administration.

FIG. 20 is a graph showing the results of evaluating the levels of HBsAg, HBeAg, HBV DNA, and pgRNA upon treatment with spiperone (oral administration) in an in vivo system.

FIG. 21 illustrates the results of IHC staining analysis for HBcAg in liver tissue upon treatment with spiperone (oral administration) in an in vivo system.

FIG. 22 illustrates the results of fluorescent immunostaining analysis for the nucleus and HBsAg in liver tissue upon treatment with spiperone (oral administration) in an in vivo system.

FIG. 23, FIG. 24, and FIG. 25 illustrate volcano plots showing the results of analyzing changes in RNA-seq-based gene expression upon treatment with spiperone.

FIG. 26 and FIG. 27 illustrate a heatmap showing the results of analyzing changes in the expression of RNA-seq-based ER stress-related genes upon treatment with spiperone, and graphs showing the results of comparing mRNA levels.

FIG. 28 and FIG. 29 illustrate heatmaps showing the results of analyzing changes in the expression of RNA-seq-based type I IFN-related genes upon treatment with spiperone, and graphs showing the results of comparing mRNA levels.

FIG. 30 illustrates the results of evaluating, by Western blot analysis, the ability of spiperone to induce ER stress through activation of the PERK-elF2α-ATF4 axis.

FIG. 31 illustrates the results of evaluating, by fluorescent immunostaining analysis, the ability of spiperone to induce ER stress through activation of the PERK-elF2α-ATF4 axis.

FIG. 32 illustrates the results of evaluating, by IHC analysis, the ability of spiperone to induce ER stress through activation of the PERK-elF2α-ATF4 axis.

FIG. 33, FIG. 34, and FIG. 35 illustrate graphs showing the results of analyzing whether spiperone treatment increases mtROS, leaks mtDNA into the cytoplasm, and induces oxidative DNA damage, and fluorescent immunostaining images.

FIG. 36 illustrates the results of evaluating, by Western blot analysis, the expression levels of proteins involved in the type I IFN activation pathway upon treatment with spiperone.

FIG. 37 illustrates graphs showing the results of analyzing the dependence of elF2a and IFI16 on the type I IFN induction mechanism by spiperone.

FIG. 38 illustrates an in vivo experimental schedule for evaluating the dependence of the anti-HBV effect of spiperone on type I IFN.

FIG. 39 illustrates graphs showing the results of evaluating the levels of HBsAg, HBeAg, HBV DNA, liver pgRNA AND liver cccDNA to evaluate the dependence of spiperone on the type I IFN pathway by using IFNAR knockout mice.

FIG. 40 illustrates the results of fluorescent immunostaining analysis to evaluate the dependence of spiperone on the type I IFN pathway by using IFNAR knockout mice.

FIG. 41 illustrates the results of IHC staining analysis to evaluate the dependence of spiperone on the type I IFN pathway by using IFNAR knockout mice.

FIG. 42 illustrates the results of evaluating the epigenetic inhibitory mechanism by which spiperone acts on HBV cccDNA, by ChIP assays.

FIG. 43 is a schematic view of evaluating the ability of spiperone to reduce HBV cccDNA in an AAV-HBV mouse model (in vivo).

FIG. 44 illustrates an in vivo experimental schedule for evaluating the ability of spiperone to inhibit cccDNA.

FIG. 45 illustrates graphs showing the results of evaluating the levels of HBsAg, HBeAg, HBV DNA, and pgRNA to evaluate the anti-HBV activity of spiperone in an in vivo cccDNA mouse model.

FIG. 46 illustrates the results of fluorescence immunostaining analysis to evaluate the anti-HBV activity of spiperone in an in vivo cccDNA mouse model.

FIG. 47 illustrates the results of IHC staining analysis to evaluate the anti-HBV activity of spiperone in an in vivo cccDNA mouse model.

FIG. 48 illustrates the results of RT-qPCR quantitative analysis to evaluate the cccDNA inhibitory effect of spiperone by using an in vivo cccDNA mouse model.

FIG. 49 illustrates the results of Southern blot analysis to evaluate the cccDNA inhibitory effect of spiperone by using an in vivo cccDNA mouse model.

FIG. 50 illustrates the results of cccDNA-selective PCR electrophoresis to evaluate the cccDNA inhibitory effect of spiperone by using an in vivo cccDNA mouse model.

### MODE FOR THE INVENTION

Hereinafter, preferred examples will be presented to aid in understanding of the present disclosure. However, these examples are provided only to facilitate the understanding of the present disclosure and are not intended to limit the scope of the present disclosure. Embodiments may allow for various modifications, and thus are not limited by the examples disclosed below and may be embodied in various forms.

The terms or words used in the specification and claims of the present disclosure should not be construed as being limited to ordinary or dictionary meanings and should be construed as meanings and concepts consistent with the technical spirit of the present disclosure based on a principle that an inventor can appropriately define concepts of terms to explain the disclosure of the inventor in the best way.

Throughout the specification of the present disclosure, when a portion is referred to as “comprising” a certain element, this does not mean that other elements are excluded, but rather means that other elements may further be included, unless specifically stated otherwise.

In the entire specification, “A and/or B” means A or B, or A and B.

The research for the present disclosure was conducted using a compound library provided by the Korea Chemical Bank.

The research outcomes of the present disclosure were achieved through research supported by the National Research Foundation of Korea (NRF) with funding from the government (Ministry of Science and ICT) (Project No.: 2710079365).

The research outcomes of the present disclosure were achieved through research supported by the Korea Health Industry Development Institute (KHIDI) with funding from the Ministry of Health and Welfare (Project No.: 2460002949).

### Example 1. Evaluation of Antiviral Activity and Safety of Spiperone (In Vitro)

The antiviral activity of spiperone was evaluated by assessing various indicators of the HBV virus, including HBV viral DNA levels, HBsAg and HBeAg levels, pgRNA levels, cccDNA levels, and Capsid protein levels, and the stability of spiperone was evaluated by assessing cytotoxicity.

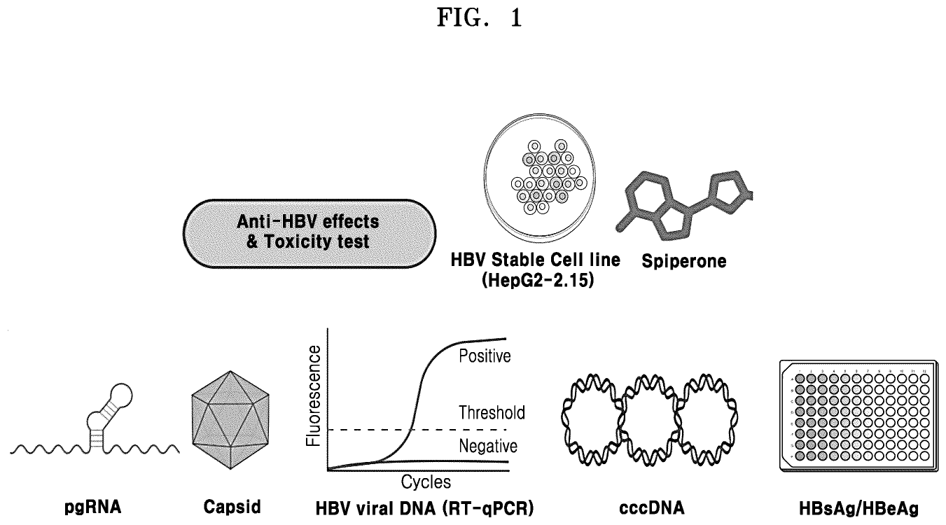

FIG. 1 is a schematic view for evaluating the antiviral activity of spiperone (in vitro).

### 1.1 Evaluation of Safety of Spiperone

HepG2.2.15, which is a subtype of HepG2 (human hepatocellular carcinoma) that stably expresses HBV, was treated with spiperone (SPP) to analyze cytotoxicity through lactate dehydrogenase assay (LDH assay) (OD 492 nm).

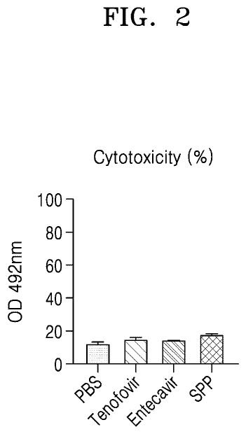

FIG. 2 is a graph for evaluating the cytotoxicity of spiperone.

As illustrated in FIG. 2, spiperone exhibited cytotoxicity at a level similar to a control (PBS), and exhibited cytotoxicity at levels similar to entecavir (ETV) and tenofovir (TDF), which are existing antiviral agents.

1.2 Measurement of HBsAg, HBeAg, HBV DNA, and HBV pgRNA Levels Upon Treatment with Spiperone in HBV Stable Cell Lines and HBV Injection System

To evaluate the antiviral effect of spiperone, in vitro experiments were performed using HepG2.2.15 and an HBV virion-infected HepG2-NTCP-C4 cell line.

HepG2.2.15 is a cell line in which the HBV genome is stably integrated into the human hepatoma cell line HepG2, enabling continuous HBV replication and viral antigen expression. The corresponding cell line was treated with spiperone, and then the viral inhibitory ability thereof was compared and analyzed with that of tenofovir (TDF), which is an existing commercially available antiviral agent.

In addition, HepG2-NTCP-C4 is a cell line that enables infection with HBV virions through overexpression of an NTCP receptor on the cell membrane of HepG2, and is widely used in liver disease research due to the highest similarity thereof to human liver cells. Accordingly, the viral inhibitory ability was compared and analyzed with that of tenofovir, which is an existing commercially available antiviral agent, 5 to 7 days after treating the HBV virion-infected HepG2-NTCP-C4 cell line with spiperone.

Specifically, the levels of HBsAg and HBeAg in cell culture media treated with spiperone were measured by using HBsAg ELISA kit (KA0289) and HBeAg ELISA kit (NBP2-60029-1), and the OD values were read by using TECAN equipment.

For quantification of HBV DNA, HBV DNA was extracted from cells using a Qiagen blood extraction kit, and DNA levels were measured through RT-qPCR analysis.

Also, to measure pgRNA levels upon treatment with spiperone, cell pellets were obtained and total RNA was extracted using TRIzol reagent (Invitrogen, Carlsbad, CA, United States). The extracted RNA samples were incubated at 37° C. for 60 minutes using RQ1 DNase (Promega, United Kingdom), and then mixed with 1 μl of a stop solution and left at 65° C. for 10 minutes to inactivate DNAse. The RNA samples were stored at −80° C., and 1 μg of RNA was reverse-transcribed using the Reverse Transcription System (Promega, United Kingdom) to measure viral pgRNA. 2 μl of the produced cDNA was used for RT-qPCR analysis, and GAPDH was used as an internal control gene for quantification.

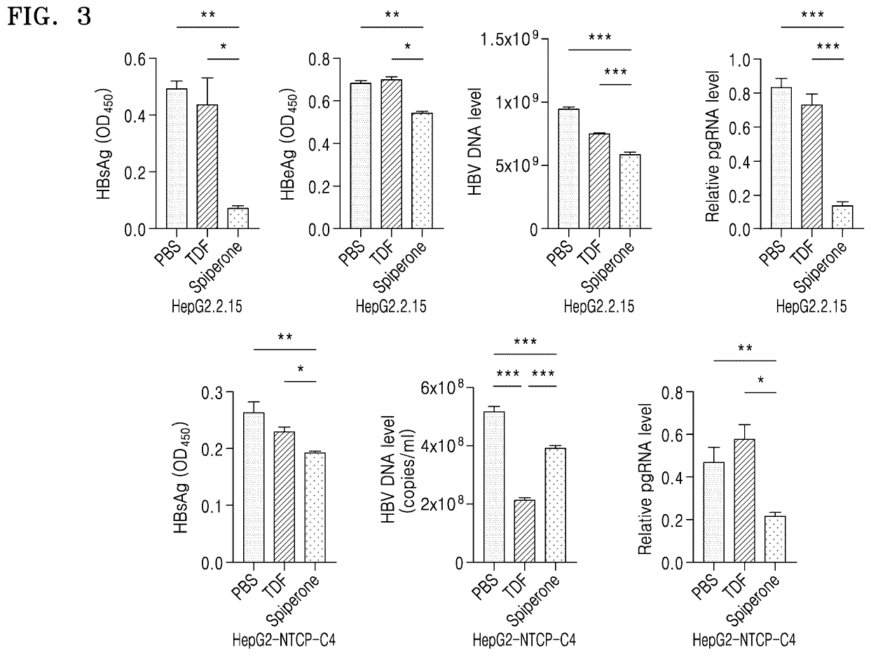

FIG. 3 is a graph showing the results of evaluating the levels of HBsAg, HBeAg, HBV DNA, and pgRNA upon treatment with spiperone in an in vitro system.

As illustrated in FIG. 3, it was confirmed that, as a result of conducting in vitro experiments using HepG2.2.15 and an HBV virion-infected HepG2-NTCP-C4 cell line to evaluate the antiviral effect of spiperone, the expression of HBsAg and HBeAg was significantly reduced in the spiperone-treated groups and the HBV DNA levels were also reduced, compared with tenofovir (TDF), which is an existing antiviral agent. Moreover, pgRNA, which is a key indicator reflecting the transcription and replication activity of HBV, was also significantly reduced upon treatment with spiperone, compared to a control.

1.3 Identification of HBV Structural Protein, HBcAg, and Capsid Levels Upon Treatment with Spiperone

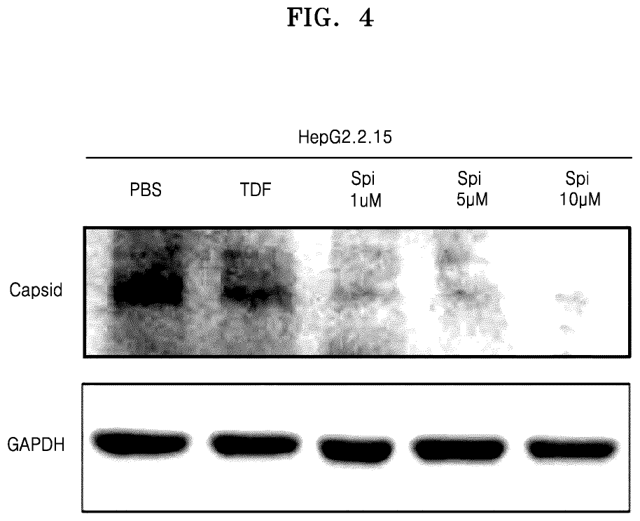

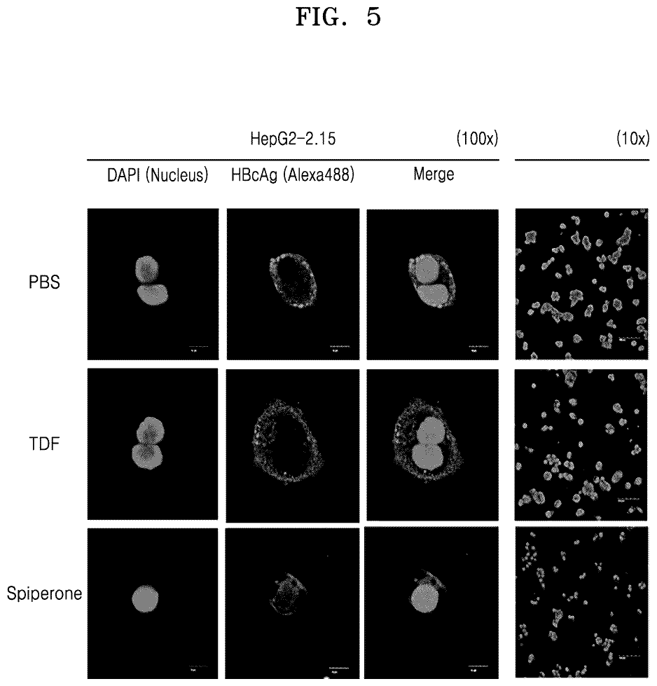

HepG2.2.15 cells were treated with spiperone, and then cultured for 24 to 48 hours, and the amount of Capsid protein in a culture medium was confirmed by Western blot analysis and confocal analysis using Anti-Hepatitis B Virus Core Antigen antibody (Abcam, #ab8638). The results thereof are shown in FIGS. 4 and 5.

FIG. 4 illustrates the results of measuring the levels of Capsid upon treatment with spiperone in an in vitro system by Western blot analysis.

FIG. 5 illustrates the results of measuring the levels of HBcAg and Capsid upon treatment with spiperone in an in vitro system by Confocal analysis.

As illustrated in FIG. 4, it was confirmed through Western blotting that the expression of Capsid was reduced in a concentration-dependent manner in the spiperone-treated groups, and was also reduced compared with the tenofovir (TDF)-treated groups.

As illustrated in FIG. 5, the confocal analysis results showed a remarkable reduction in the intracellular fluorescence signal of HBcAg upon treatment with spiperone compared with PBS and the TDF-treated groups, through which it was confirmed that spiperone effectively inhibits HBV capsid protein expression and core particle formation.

1.4 Confirmation of Level of cccDNA as Template for HBV Replication and Key To Infection

To evaluate the inhibitory effect of spiperone against HBV cccDNA, cccDNA was purified from HepG2.2.15 and HepG2-NTCP-C4, which is an HBV-infected cell line, followed by selective amplification, to perform agarose gel electrophoresis and RT-qPCR analysis.

Specifically, to measure cccDNA, linearized 1.2X genotype C2 HBV plasmid was transfected into HepG2.2.15 and HepG2-NTCP-C4 cell lines, and then lysis buffer A (10 mM Tris-HCl, pH 7.4, 1 mM EDTA, 50 mM NaCl, 1% NP-40) was added to the obtained cells and left at 4° C. for 10 minutes. Subsequently, the lysate was centrifuged at 14,000 rpm for 10 minutes to obtain a nuclear pellet portion, which was then resuspended in lysis buffer B (10 mM Tris-HCl, 10 mM EDTA, 150 mM NaCl, 0.5% SDS). Subsequently, sonication (3 to 4 pulses, 5 seconds) was performed to disrupt the nucleus, followed by addition of 0.5 mg/ml of proteinase K and incubation at 37° C. overnight.

The next day, DNA was extracted from the lysate through phenol-chloroform (1:1) treatment and ethanol precipitation, and 1 μg of the extracted DNA was treated with 10 U of PSAD (Plasmid Safe DNase I, Epicenter, PA, United States) at 37° C. for 45 minutes. Thereafter, the enzyme activity was stopped at 70° C. to remove linear DNA, thereby purifying only cccDNA. The obtained cccDNA was quantified by RT-qPCR using cccDNA primers [forward: 5′-CCG TGT GCA CTT CGC TTC A-3′ (SEQ ID NO: 1) and reverse: 5′-GCA CAG CTT GGA GGC TTG A-3′ (SEQ ID NO: 2)], and mtDNA was used as an internal control.

HBV cccDNA was amplified using cccDNA-selective primers [forward: 5′-GGG GCG CAC CTC TCT TTA-3′ (position 1523-1540) (SEQ ID NO: 3) and reverse: 5′-AGG CAC AGC TTG GAG GC-3′ (position 1886-1870) (SEQ ID NO: 4)] and cccDNA-nonselective primers [forward: 5′-CAC TCT ATG GAA GGC GGG TA-3′ (position 2755-2774) (SEQ ID NO: 5) and reverse: 5′-TGC TCC AGC TCC TAC CTT GT-3′ (position 3002-2983) (SEQ ID NO: 6)], and the amplified products were detected through 1% agarose gel electrophoresis.

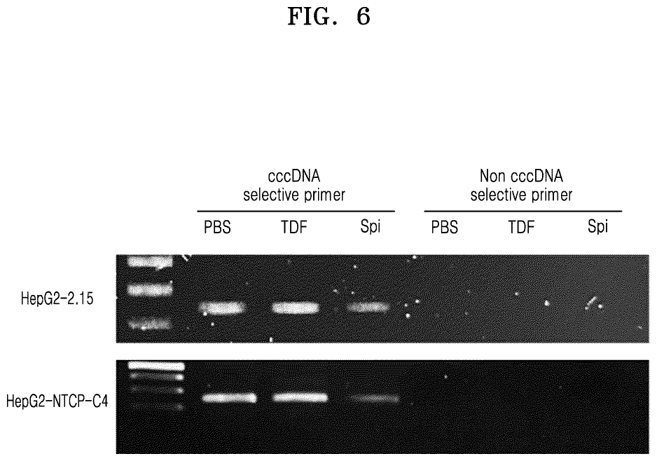

FIG. 6 illustrates the results of measuring cccDNA levels upon treatment with spiperone in an in vitro system by agarose gel electrophoresis analysis.

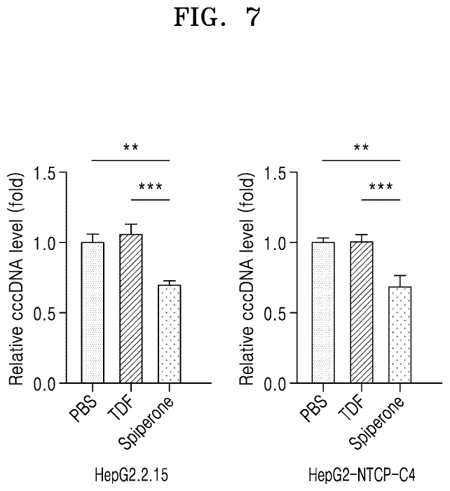

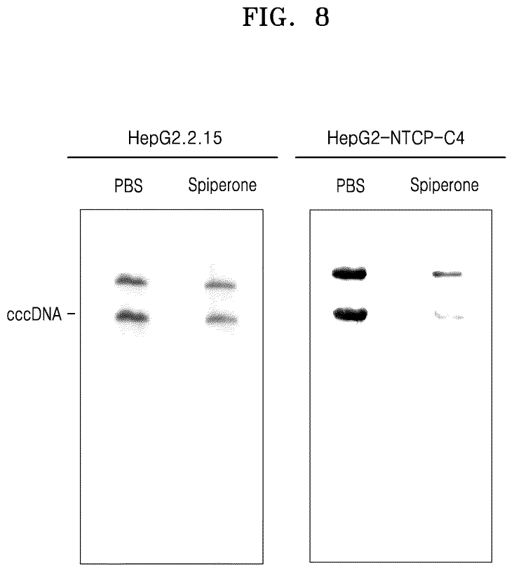

FIG. 7 illustrates the results of measuring cccDNA levels upon treatment with spiperone in an in vitro system by RT-qPCR analysis.

As illustrated in FIG. 6, the electrophoresis analysis results showed that cccDNA band intensity was significantly reduced in the spiperone-treated groups compared to PBS- and TDF-treated groups.

As illustrated in FIG. 7, the RT-qPCR results confirmed a significant reduction in cccDNA level only in the spiperone-treated groups.

FIG. 8 illustrates Southern Blot images showing cccDNA band in HepG2.2.15 and HepG2-NTCP-C4 cells treated with PBS or Spiperone.

As illustrated in FIG. 8, Southern blotting confirmed a reduction in the cccDNA band in Spiperone treated cells.

Also, after HepG2.2.15 cells were treated with PBS, IFN-α, or spiperon, HBsAg levels were analyzed by ELISA, and cccDNA levels were analyzed by qPCR and agarose gel electrophoresis.

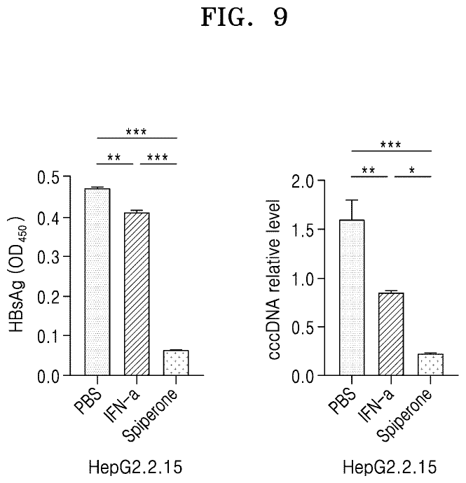

FIG. 9 illustrates the results of measuring HBsAg levels and cccDNA levels upon treatment with spiperone in an in vitro system by ELISA and qPCR analysis.

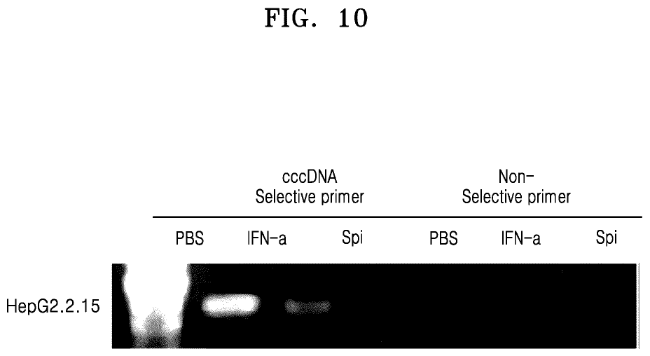

FIG. 10 illustrates the results of measuring cccDNA levels upon treatment with spiperone in an in vitro system by agarose gel electrophoresis analysis.

As illustrated in FIG. 9, the suppression of HBsAg secretion (p<0.001) and cccDNA levels (p<0.001) by Spiperone more efficiently than IFN-α.

As illustrated in FIG. 10, the electrophoresis analysis results showed that cccDNA band intensity was significantly reduced in the spiperone-treated groups compared to PBS- and IFN-α-treated groups.

Also, after HBV WT virion infection in HepaRG, PBS, TDF, or spiperon were treated, and 5 days later, HBsAg, BeAg ELISA, and HBV DNA levels were confirmed using the supernatant (Day 5 sup), and pgRNA and cccDNA levels were measured using the cell pellet in an in vitro system by ELISA and qPCR analysis.

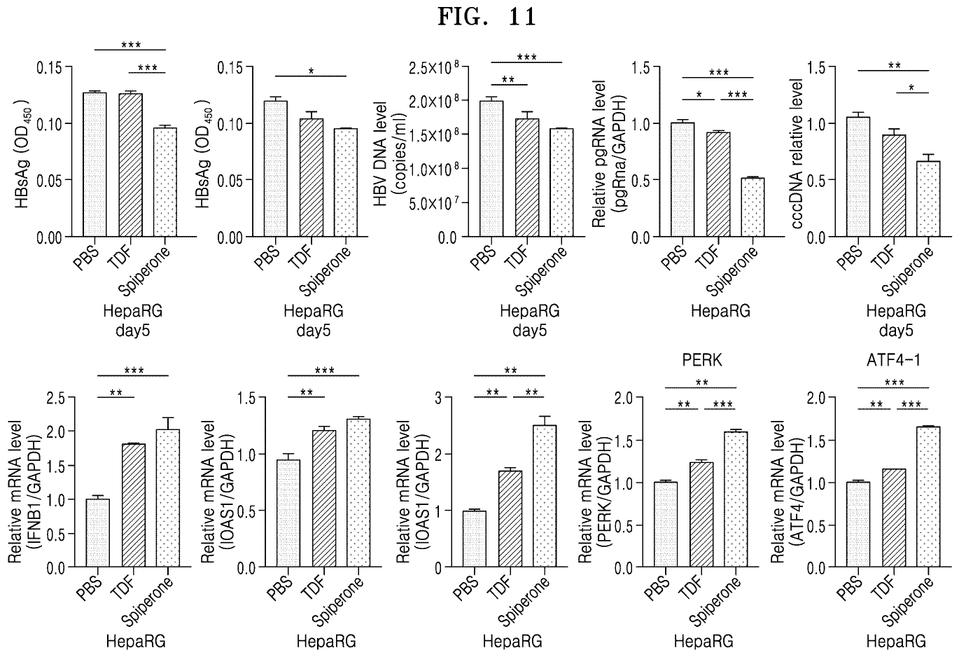

FIG. 11 illustrates the results of HBV antiviral efficacy using HepaRG.

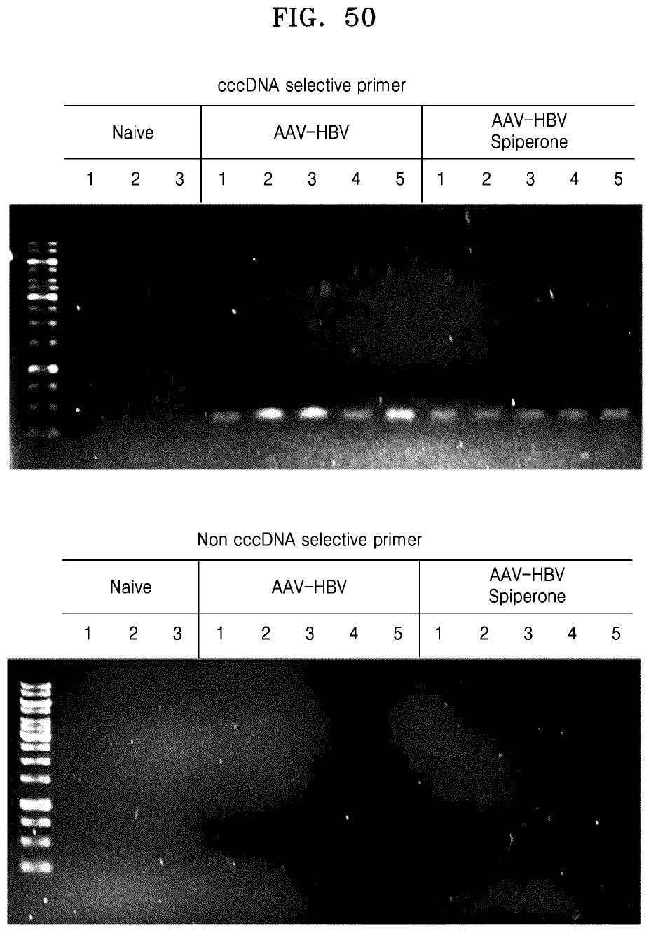

As illustrated in FIG. 50, the HBsAg and HBV DNA secretion of supernatant, the pgRNA and cccDNA levels of the cell pellet were significantly reduced in the spiperone-treated groups compared to PBS- and TDF-treated groups.

### Example 2. Evaluation of Synergistic Effect of Spiperone and Antiviral Agent

Synergy Finder was used to evaluate the synergistic effect of spiperone and an antiviral drug currently used in clinical practice.

Specifically, HepG2.2.15 cells were treated with spiperone and tenofovir (TDF) at different concentrations, and the levels of HBsAg in the culture media were measured through ELISA after 48 hours. Then, synergy scores were calculated through the Synergy finder program. Synergistic effects were considered to exist when the synergy score was 5 or higher using the ZIP calculation method.

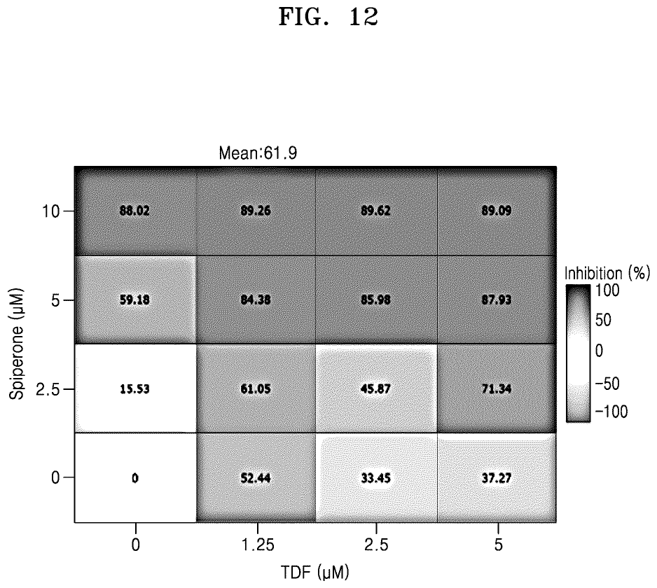

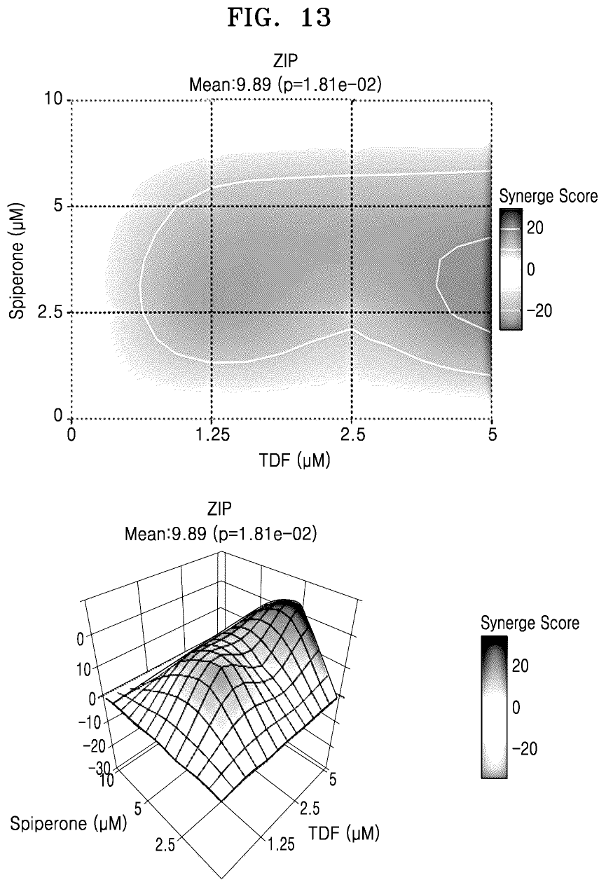

FIGS. 12 and 13 illustrate the results of evaluating the synergistic effect of spiperone and tenofovir.

As illustrated in FIGS. 12 and 13, when HepG2.2.15 cells were treated with spiperone and TDF in combination, a high score of 15 points or more was confirmed, confirming a high synergistic effect.

Through this synergistic effect of spiperone and the antiviral agent, it can be concluded that the combination of spiperone in administering an antiviral agent or a therapeutic agent for viral infection, such as applying spiperone as an additional drug for HBV patients currently using an existing nucleotide analogue antiviral agent (e.g., TDF), would be highly advantageously applicable in clinical practice.

### Example 3. Evaluation of Antiviral Activity of Spiperone (In Vivo)

Spiperone and a standard drug were injected into HBV transgenic mice (C57B1/6N mice expressing pHY92-1.1x-HBV-full genome plasmid), and then serum was isolated and obtained through orbital blood collection, and HBV DNA, HBeAg, and HBsAg levels in the serum were measured through RT-PCR or ELISA. In addition, the anti-HBV activity of spiperone in the mouse model was evaluated by analyzing viral pgRNA levels in mouse liver tissues.

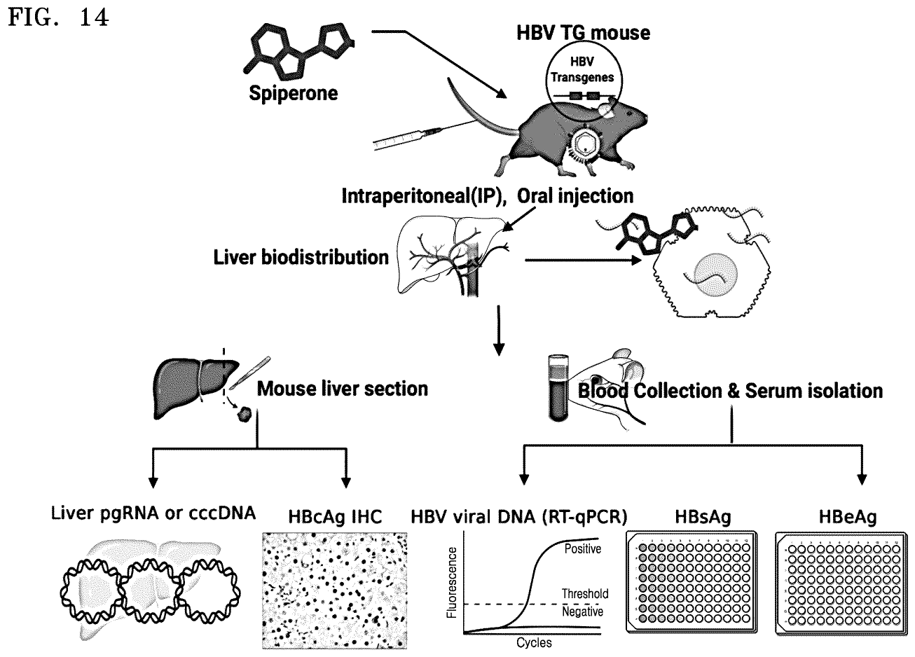

FIG. 14 is a schematic view for evaluating the antiviral activity of spiperone (in vivo).

### 3.1 Confirmation of Antiviral Activity of Spiperone Upon Intraperitoneal Injection (Ip Injection)

To evaluate the antiviral activity of spiperone in HBV transgenic (C57BL/6) mice, PBS, tenofovir (TDF) (500 μg/mouse), and spiperone (1.5 mg/kg) were intraperitoneally administered daily for 8 weeks.

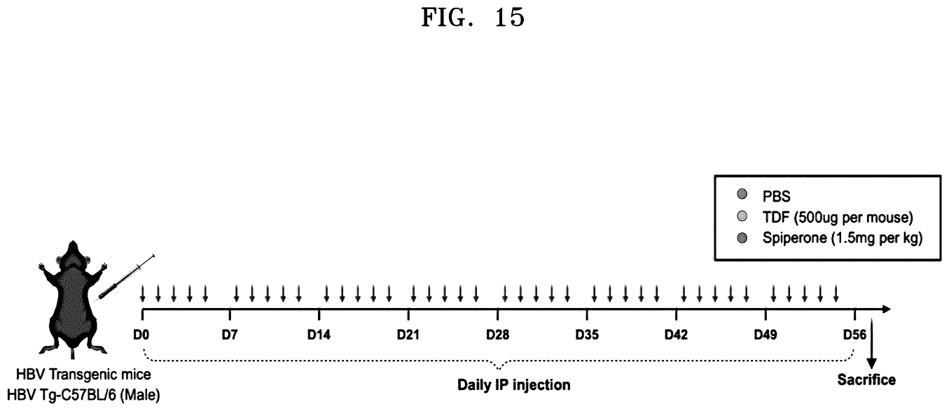

FIG. 15 illustrates an in vivo experimental schedule for evaluating the antiviral activity and viral infection treatment efficacy of spiperone upon intraperitoneal injection.

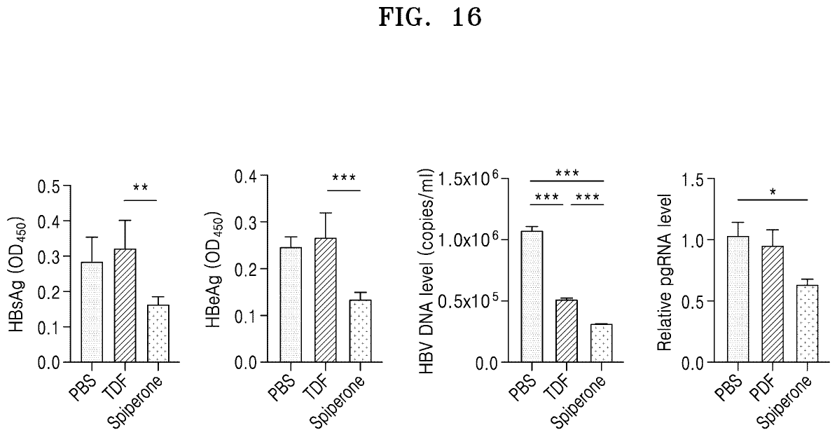

The experimental results thereof are shown in FIGS. 16 to 18.

FIG. 16 is a graph showing the results of evaluating the levels of HBsAg, HBeAg, HBV DNA, and pgRNA upon treatment with spiperone (intraperitoneal injection) in an in vivo system.

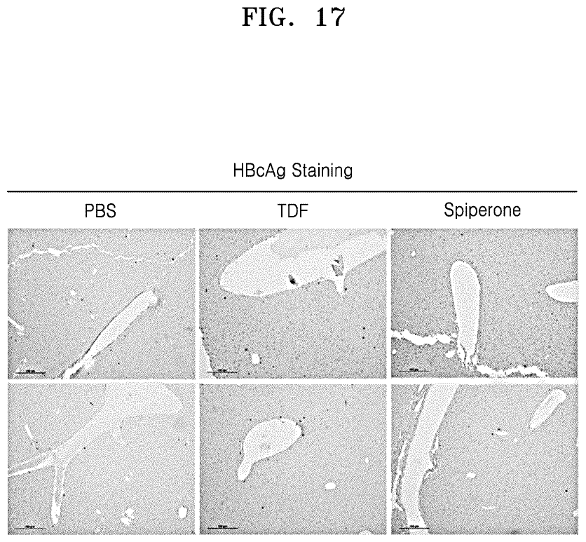

FIG. 17 illustrates the results of immunohistochemical (IHC) staining analysis for HBcAg in liver tissue upon treatment with spiperone (intraperitoneal injection) in an in vivo system.

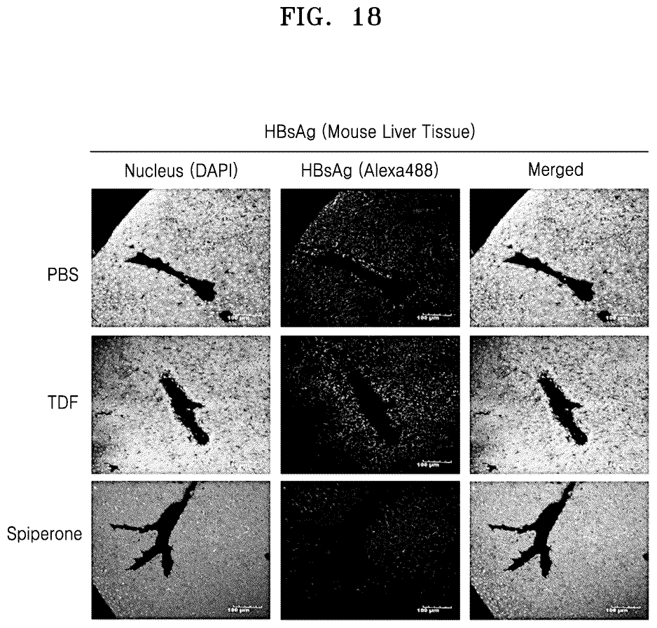

FIG. 18 illustrates the results of fluorescent immunostaining analysis for the nucleus and HBsAg in liver tissue upon treatment with spiperone (intraperitoneal injection) in an in vivo system.

As illustrated in FIGS. 16 to 18, the serum analysis results showed that HBsAg, HBeAg, and HBV DNA levels in the spiperone-treated groups were significantly reduced compared to a control (PBS) and tenofovir (TDF)-treated groups, and the level of pgRNA expression in the liver was also significantly inhibited. In the immunohistochemical (IHC) staining for HBcAg in liver tissue, a significant reduction in HBV-infected cells was confirmed in the spiperone-treated groups. Similarly, fluorescence immunostaining for HBsAg visually demonstrated a significant decrease in HBV antigen expression in liver tissue in the spiperone-treated groups.

### 3.2 Confirmation of Antiviral Activity of Spiperone Upon Oral Administration (Oral Gavage)

To evaluate the antiviral activity of spiperone in HBV transgenic (C57BL/6) mice upon oral administration, PBS, tenofovir (TDF) (500 μg/mouse), and spiperone (1.5 mg/kg) were orally administered daily for 8 weeks.

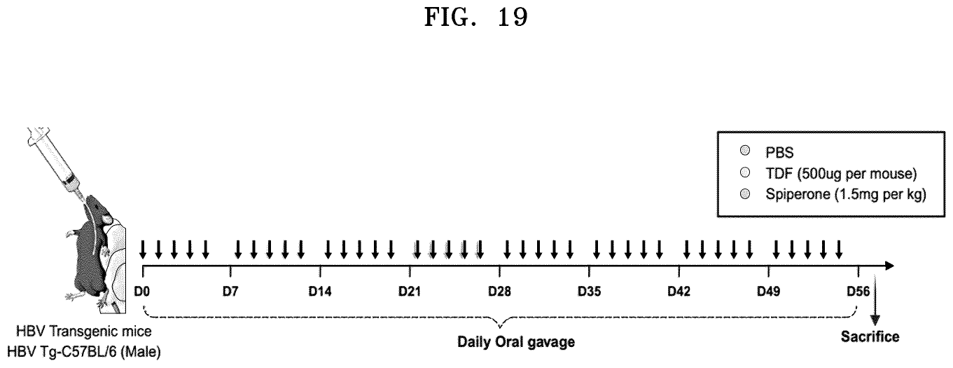

FIG. 19 illustrates an in vivo experimental schedule for evaluating the antiviral activity and viral infection treatment efficacy of spiperone upon oral administration.

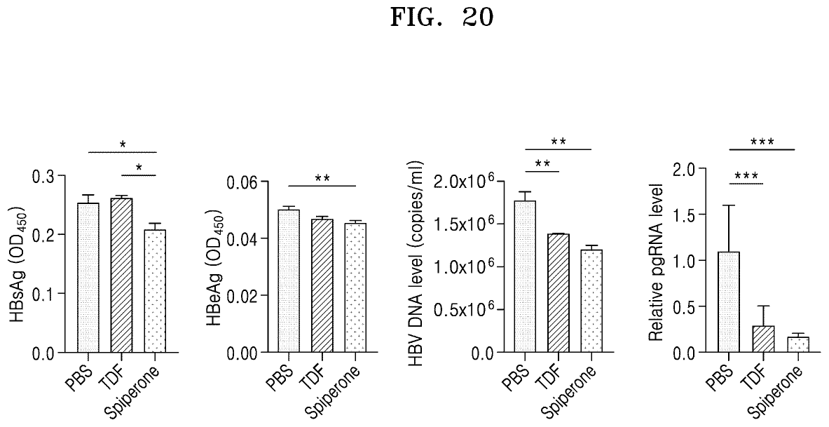

The experimental results thereof are shown in FIGS. 20 to 22.

FIG. 20 is a graph showing the results of evaluating the levels of HBsAg, HBeAg, HBV DNA, and pgRNA upon treatment with spiperone (oral administration) in an in vivo system.

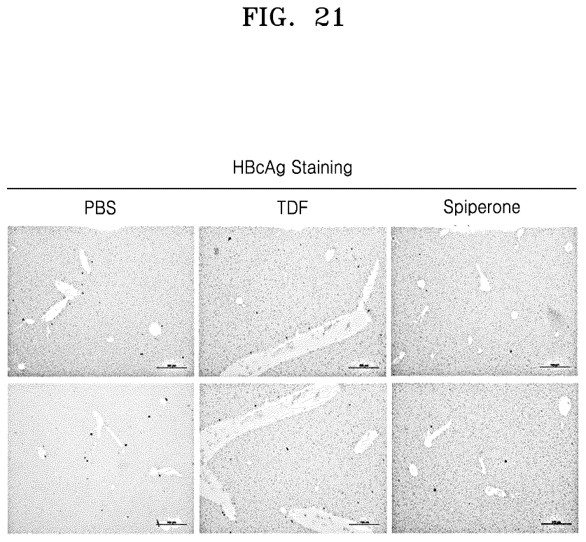

FIG. 21J illustrates the results of IHC staining analysis for HBcAg in liver tissue upon treatment with spiperone (oral administration) in an in vivo system.

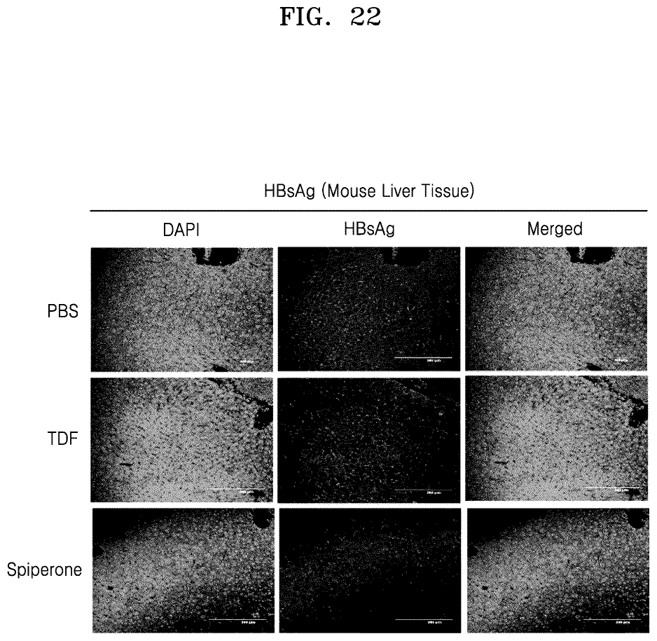

FIG. 22 illustrates the results of fluorescent immunostaining analysis for the nucleus and HBsAg in liver tissue upon treatment with spiperone (oral administration) in an in vivo system.

As shown in FIGS. 20 to 22, the serum analysis results showed that the levels of HBsAg and HBeAg were significantly reduced in the spiperone-treated groups compared to the control (PBS) and tenofovir (TDF)-treated groups, and HBV DNA and pgRNA expression levels in the liver were also significantly inhibited. In the HBcAg immunohistochemical staining (IHC) for HBcAg in liver tissue, a significant reduction in HBcAg count was confirmed in the spiperone-administered group, demonstrating a remarkable decrease in HBV-infected cells. Similarly, the HBsAg fluorescent immunostaining results showed a remarkable decrease in fluorescence intensity in the spiperone-administered groups, visually confirming a significant reduction in HBV antigen expression in the liver tissue.

### Example 4. Analysis of Mechanisms for Antiviral Activity and cccDNA Inhibitory Activity of Spiperone

4.1 Analysis of RNA-Seq and mRNA Levels

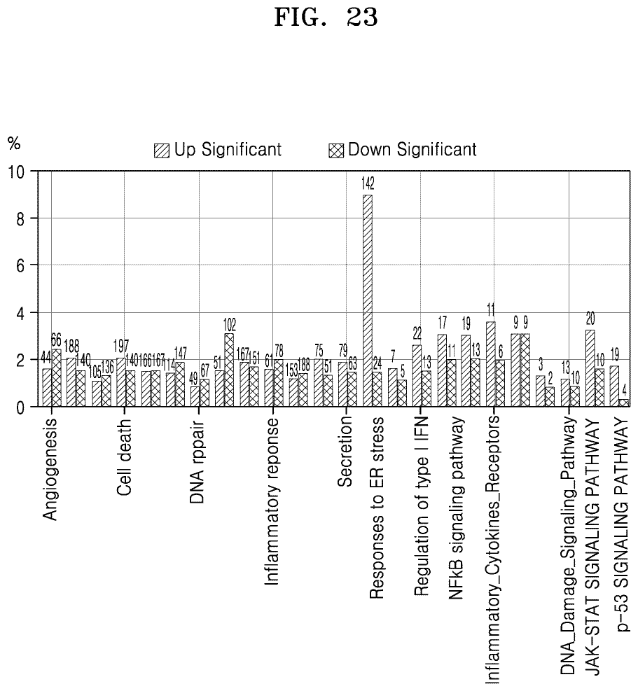

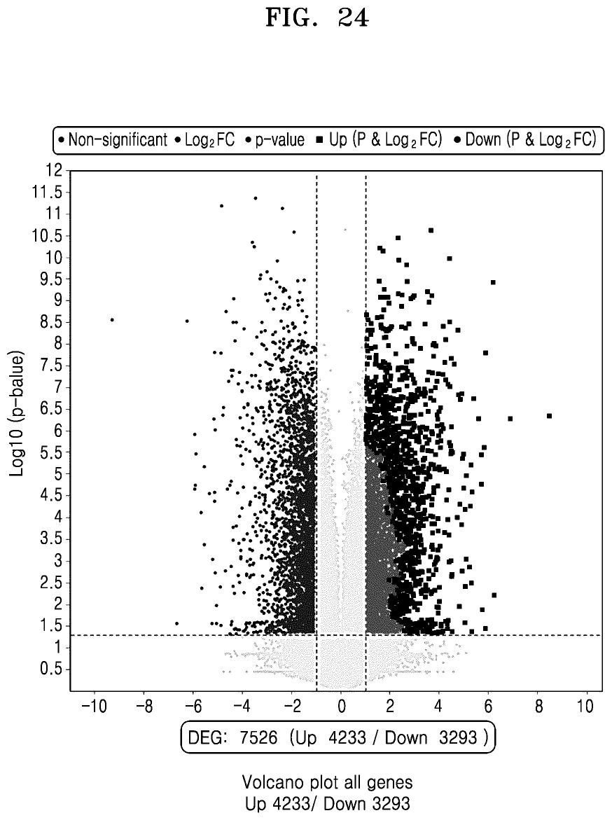

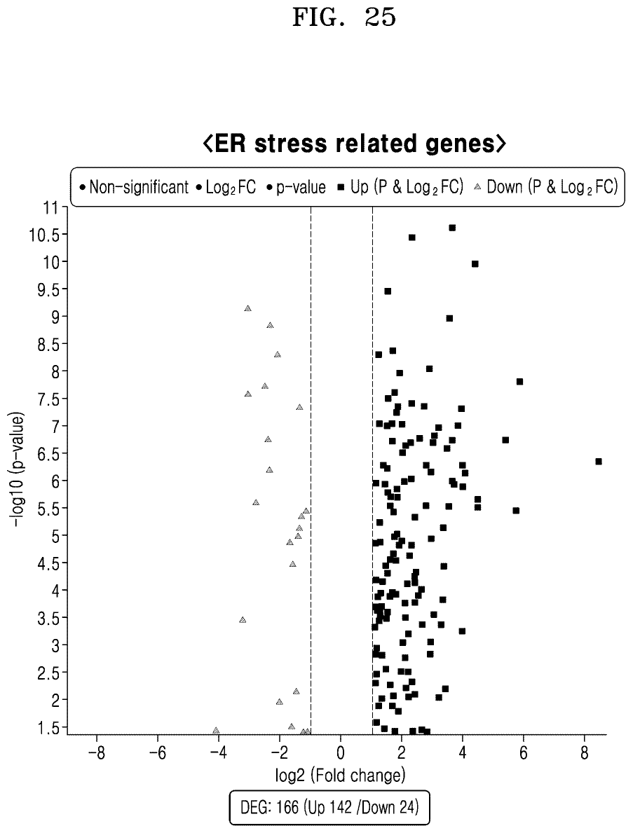

RNA-sequencing analysis was performed using HBV virion-infected HepG2-NTCP-C4 cells 12 hours after HBV virion infection. The experimental results thereof are shown in FIGS. 23 to 29.

FIGS. 23 to 25 illustrate pathway enrichment analysis and volcano plots showing the results of analyzing changes in RNA-seq-based gene expression upon treatment with spiperone.

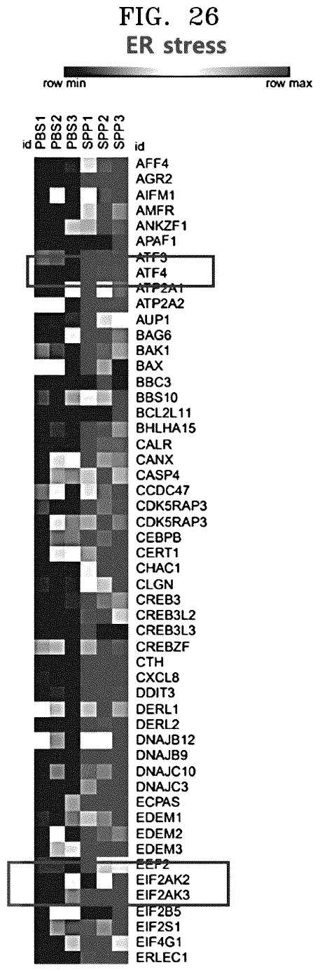

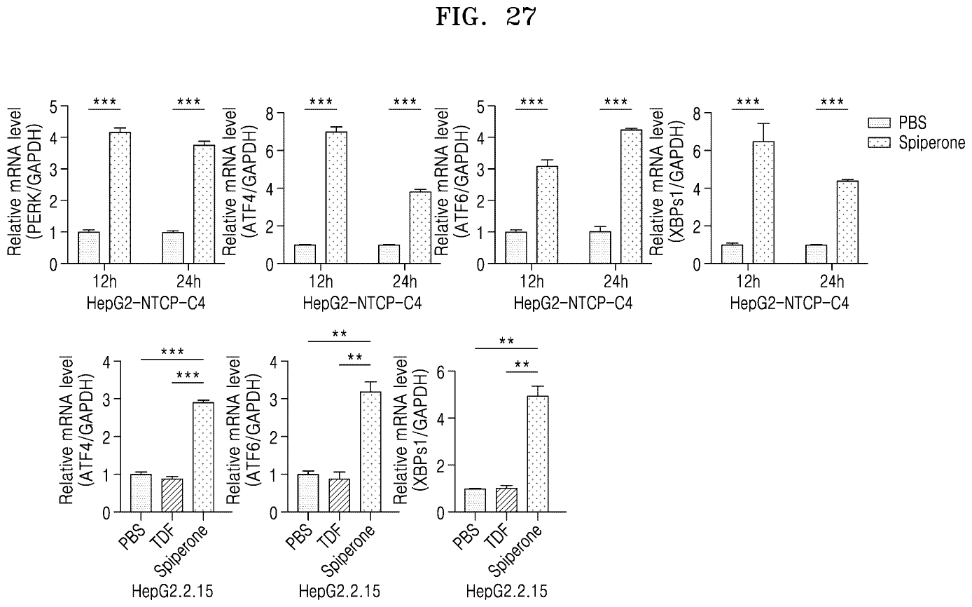

FIGS. 26 and 27 illustrate a heatmap showing the results of analyzing changes in the expression of RNA-seq-based ER stress-related genes upon treatment with spiperone, and graphs showing the results of comparing mRNA levels.

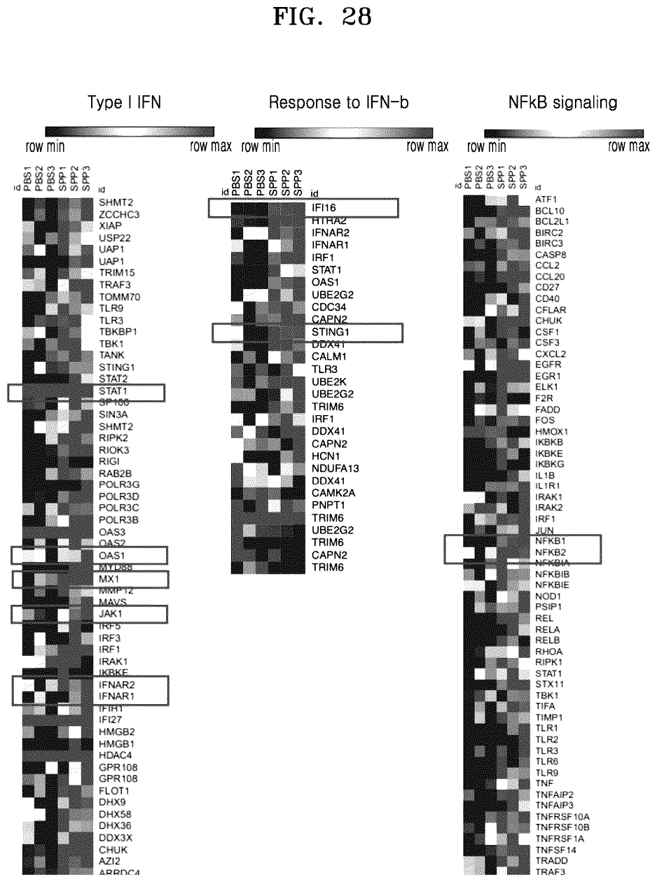

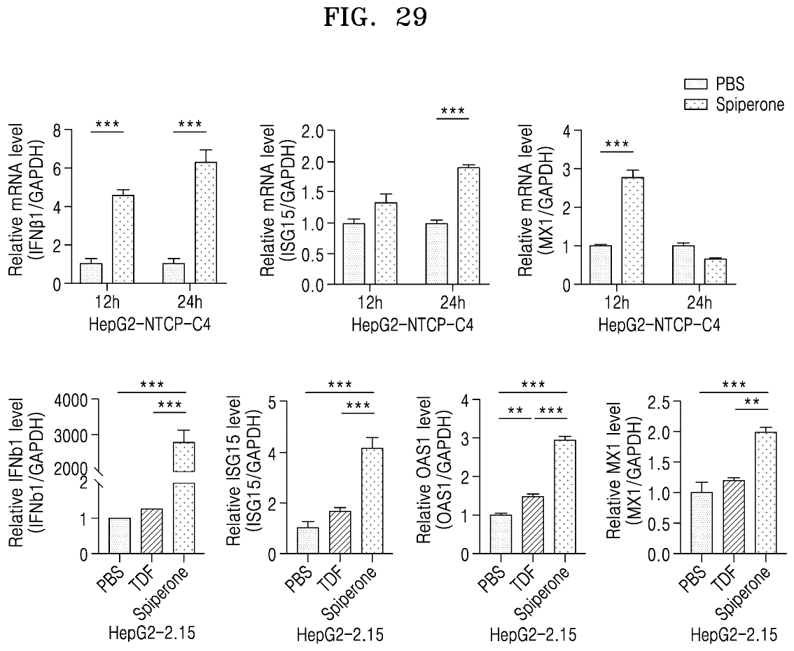

FIGS. 28 and 29 illustrate heatmaps showing the results of analyzing changes in the expression of RNA-seq-based type I IFN-related genes upon treatment with spiperone, and graphs showing the results of comparing mRNA levels.

As illustrated in FIGS. 23 to 29, the RNA-seq-based pathway enrichment analysis results confirmed that ER stress response, NF-κB signaling, and the Type I IFN-related signaling pathway were significantly activated, and in particular, the expression of ER stress-related genes was extensively increased (FIGS. 23 to 25). In addition, not only in the Volcano plot analysis but also in the heatmaps and graphs, the key ER stress pathways, including the PERK-elF2α-ATF4 axis, were strongly induced (FIGS. 26 and 27), and the expression of Type I IFN, Response to IFN-β and NF-κB-related genes (MX1, ISG15, IFNB1, OAS1, and the like) was significantly increased (FIGS. 28 and 29).

These results suggest that ER stress induced by spiperone activates the STING-TBK1-IRF3/7 pathway via cytoplasmic HBV DNA leakage and IFI16 recognition, which leads to Type I IFN secretion and interferon-stimulated gene (ISG) induction, ultimately resulting in the induction of HBV inhibition. In addition, this immune activation response may further amplify ER stress, suggesting that spiperone may have a novel mechanism of action that effectively inhibits HBV replication based on the interconnected pathway between ER stress and immune responses.

### 4.2 Verification of ER Stress-Inducing Ability of Spiperone

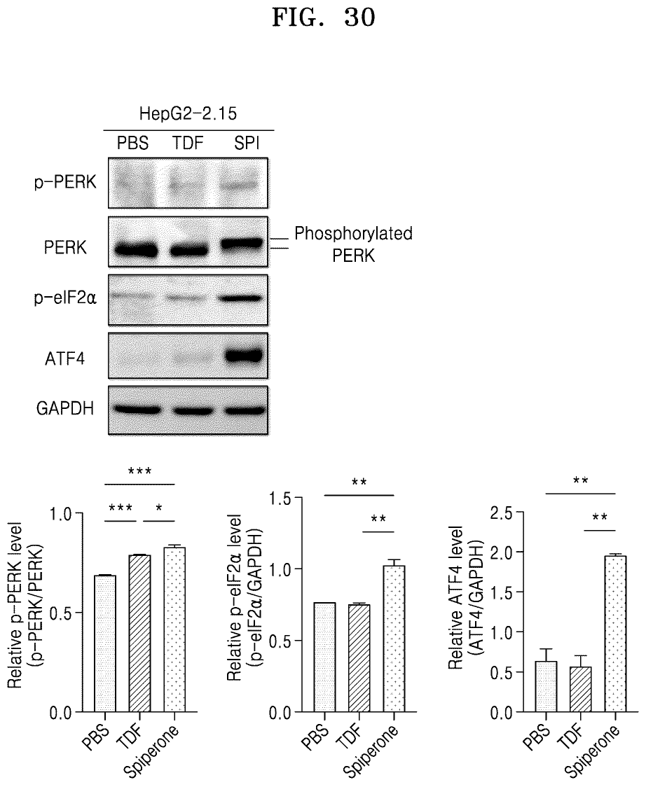

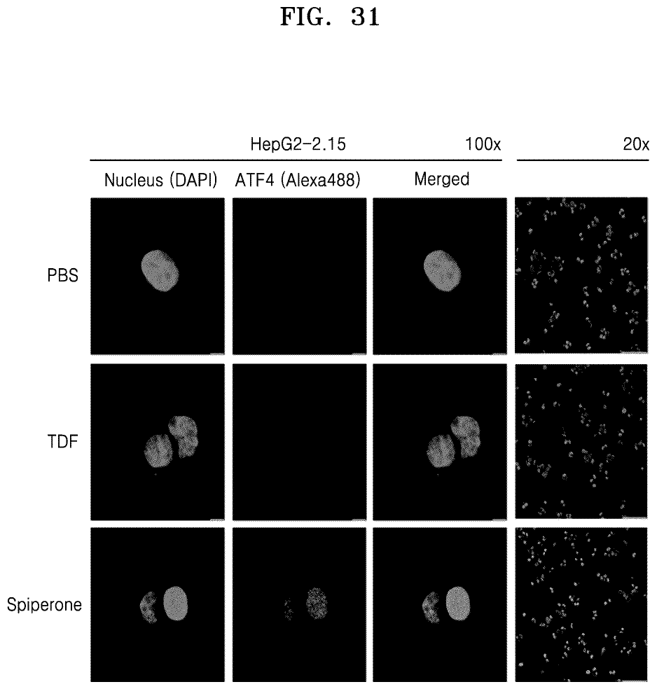

To confirm whether spiperone induces endoplasmic reticulum stress (ER stress), the expression of proteins belonging to the PERK-elF2α-ATF4 pathway was analyzed using HepG2.2.15 cells. The results thereof are shown in FIG. 30.

FIG. 30 illustrates the results of evaluating, by Western blot analysis, the ability of spiperone to induce ER stress through activation of the PERK-elF2α-ATF4 axis.

As illustrated in FIG. 30, the Western blot analysis results showed that the expression of p-PERK and p-elF2a in the spiperone-treated groups significantly increased compared to the control (PBS-treated group) and comparative groups (TDF-treated groups), and the expression of ATF4, which is a downstream transcription factor, also increased significantly. These results demonstrate that spiperone strongly induces the ER stress signaling pathway centered on the PERK axis.

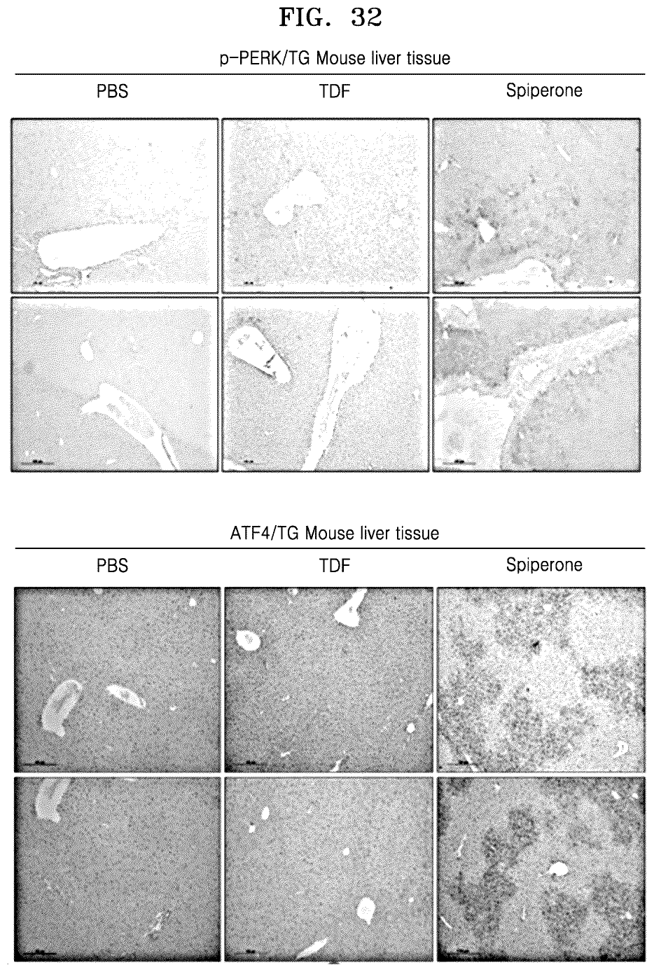

In addition, to confirm the ER stress-inducing effect of spiperone at cellular and tissue levels, the expression of ATF4 and p-PERK was analyzed through immunofluorescence staining and immunohistochemistry (IHC) staining, respectively, in HepG2.2.15 cells and HBV transgenic mice (HBV TG mice). The results thereof are shown in FIGS. 31 and 32.

FIG. 31 illustrates the results of evaluating, by fluorescent immunostaining analysis, the ability of spiperone to induce ER stress through activation of the PERK-elF2α-ATF4 axis.

FIG. 32 illustrates the results of evaluating, by IHC staining analysis, the ability of spiperone to induce ER stress through activation of the PERK-elF2α-ATF4 axis.

As illustrated in FIGS. 31 and 32, the fluorescence signal of nuclear ATF4 (Alexa Fluor 488) was remarkably increased upon treatment with spiperone in HepG2.2.15 cells compared to PBS- and TDF-treated groups, suggesting that spiperone strongly induces ATF4 expression in the cells. In addition, in mouse liver tissues, the immunoreactivity of p-PERK and ATF4 was significantly increased in the spiperone-treated groups, with strong positive signals observed throughout the liver. These results demonstrate that spiperone induces ER stress responses centered on the PERK-elF2α-ATF4 axis even at the tissue level.

4.3 Analysis of Mitochondrial Damage and mtDNA Leakage Based on ER Stress Induced by Spiperone

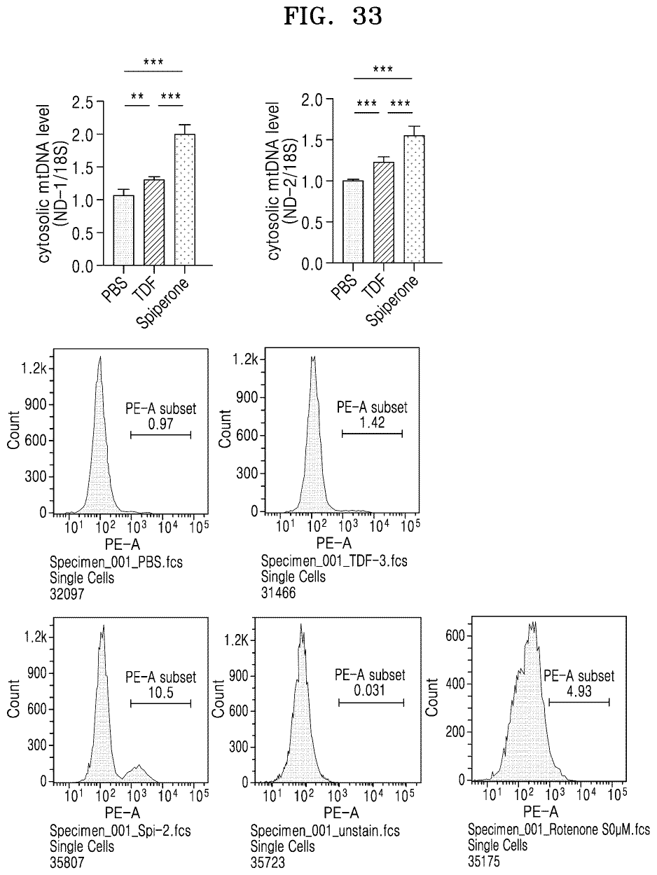

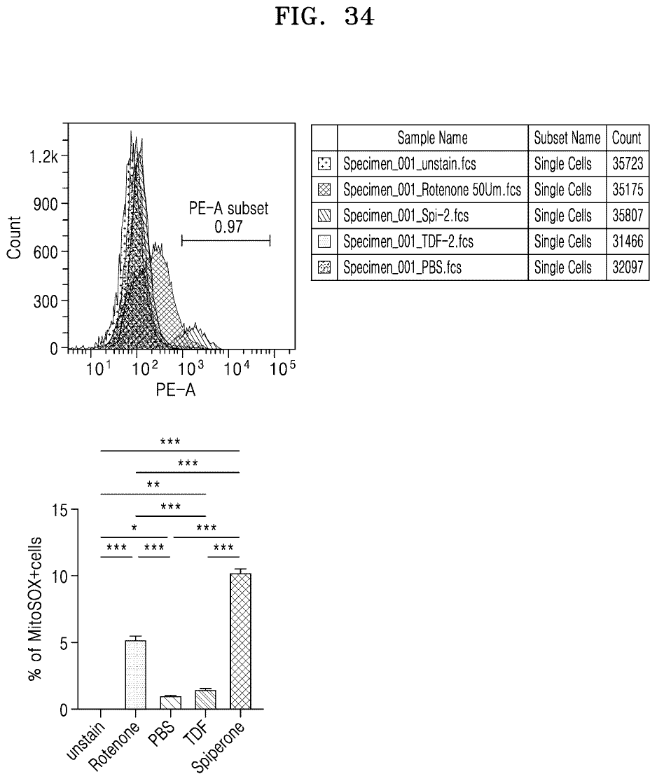

To confirm whether ER stress induced by spiperone treatment leads to mitochondrial dysfunction and oxidative stress, MitoSOX-based flow cytometry, quantitative analysis of cytosolic mitochondrial DNA (mtDNA), and immunofluorescence staining analysis of 8-hydroxy-2′-deoxyguanosine (8-OHdG), which is an oxidized DNA damage marker, were performed. The results thereof are shown in FIGS. 33 to 35.

FIGS. 33 to 35 illustrate graphs showing the results of analyzing whether spiperone treatment increases mtROS, leaks mtDNA into the cytoplasm, and induces oxidative DNA damage, and fluorescent immunostaining images.

As illustrated in FIGS. 33 to 35, the MitoSOX fluorescence signal was significantly increased in the spiperone-treated groups compared to the control (PBS) and comparative groups (TDF), suggesting a significant increase in ROS generation from mitochondria. In particular, the fluorescence signal was observed at a level similar to that of a rotenone-treated group as a positive control, confirming that spiperone induces strong mitochondrial oxidative stress.

In addition, qPCR-based quantitative analysis of cytoplasmic mtDNA showed that a ND1/18S and ND2/18S ratio was significantly increased in the spiperone-treated groups, indicating that damaged mtDNA was leaked into the cytoplasm.

According to the immunofluorescence staining analysis results, the 8-OHdG signal increased in a concentration-dependent manner as the concentration of spiperone increased, indicating that oxidized DNA was accumulated in the cells due to treatment with spiperone.

### 4.4 Confirmation of Activation of Type I Interferon (IFN) Signaling Pathway by Spiperone

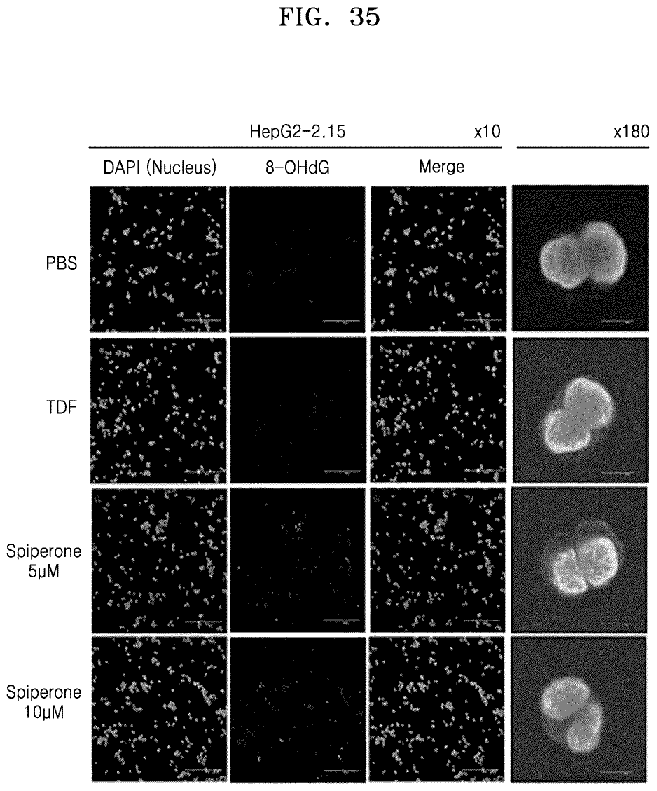

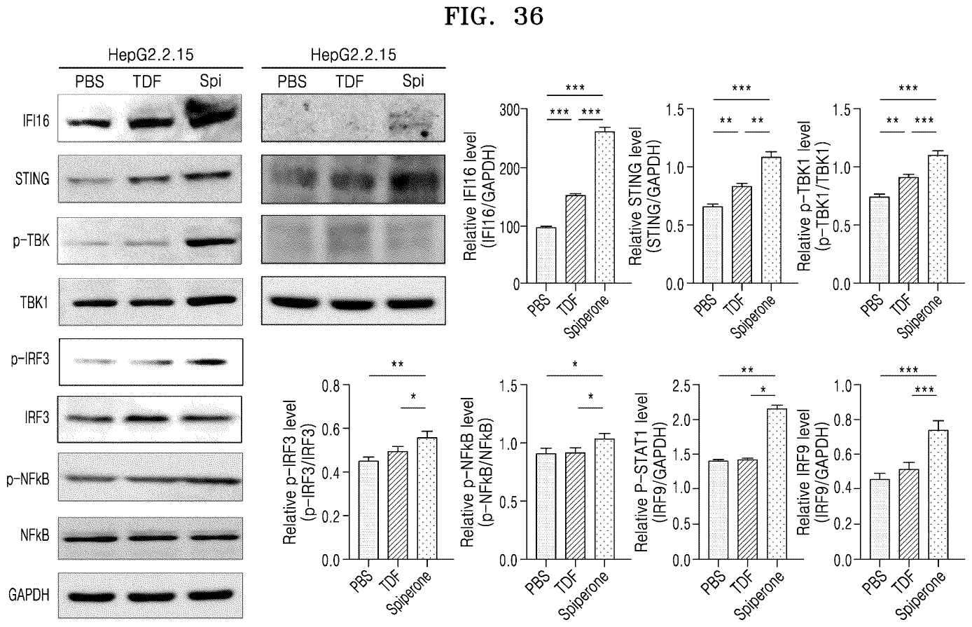

To confirm whether spiperone activates the type I IFN immune pathway, the expression and phosphorylation levels of proteins included in the IFI16-STING-TBK1-IRF3 axis, a pathway that detects damaged DNA within cells and transmits immune signals, were analyzed through Western blot. The results thereof are shown in FIG. 36.

FIG. 36 illustrates the results of evaluating, by Western blot analysis, the expression levels of proteins involved in the type I IFN activation pathway upon treatment with spiperone.

As illustrated in FIG. 36, the phosphorylation levels of proteins included in the signaling pathway were significantly increased in the spiperone-treated groups compared to the control (PBS) and comparative groups (TDF), demonstrating that spiperone can activate the type I IFN-inducing pathway for detecting damaged DNA within cells at the molecular level.

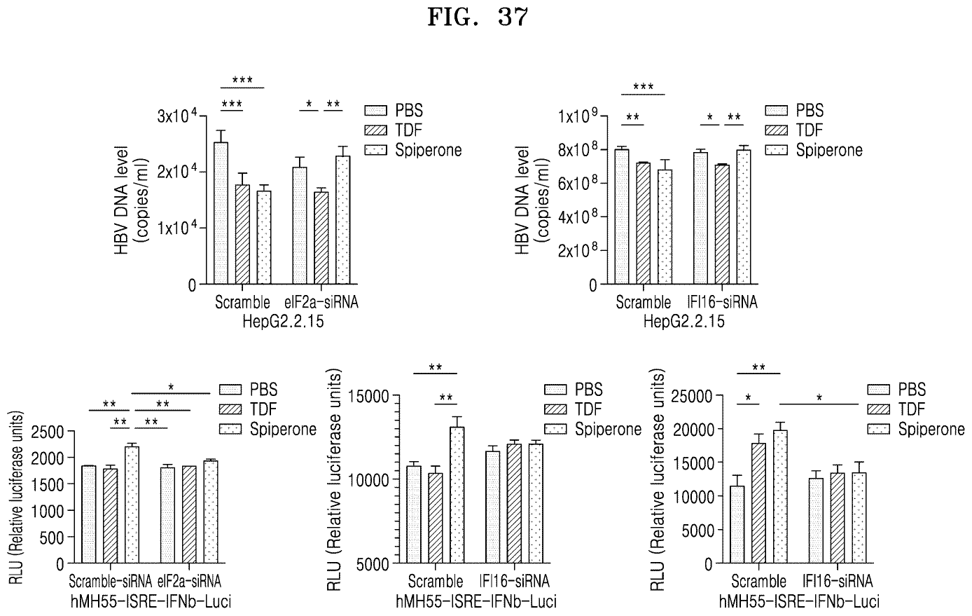

To confirm whether the antiviral effect of spiperone is related to type I IFN signaling induced by the elF2a and IFI16 pathways, HepG2.2.15 cells transfected with siRNAs for each gene were used to perform HBV DNA quantification and IFN-β promoter activity analysis using hMH55-ISRE-IFNβ-luciferase reporter cells. The results thereof are shown in FIG. 37.

FIG. 37 illustrates graphs showing the results of analyzing the dependence of elF2a and IFI16 on the type I IFN induction mechanism by spiperone.

As illustrated in FIG. 37, the HBV DNA inhibitory effect induced by spiperone was significantly decreased in the elF2α-siRNA-treated groups compared to the scramble-siRNA-treated groups, and the luciferase expression level linked to the IFNβ promoter was also reduced. Similarly, both the HBV DNA inhibitory effect and the luciferase expression were reduced under the IFI16-siRNA treatment conditions. These results demonstrate at the molecular level that spiperone exhibits antiviral activity through the ER stress and DNA sensing-based type I IFN pathway.

### Example 5. Evaluation of Dependence of Anti-HBV Effect of Spiperone on Type I IFN Using IFNAR Knockout Mice (In Vivo)

To determine whether the antiviral effect of spiperone is dependent on the type I IFN signaling pathway, C57BL/6 wild-type mice (WT) and IFNAR knockout mice (IFNAR KO) were infected with HBV via hydrodynamic injection (HDI) using pAAV-HBV1.2x plasmid, and spiperone (1.5 mg/kg) or PBS was administered intraperitoneally daily for 2 weeks, followed by analysis.

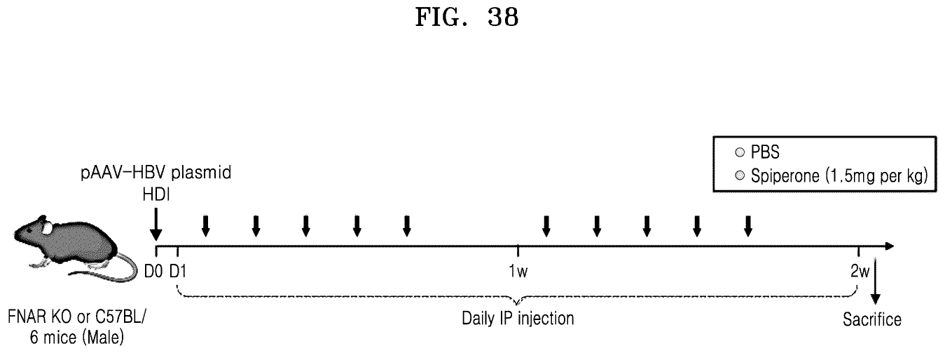

FIG. 38 illustrates an in vivo experimental schedule for evaluating the anti-HBV effect of spiperone on type I IFN.

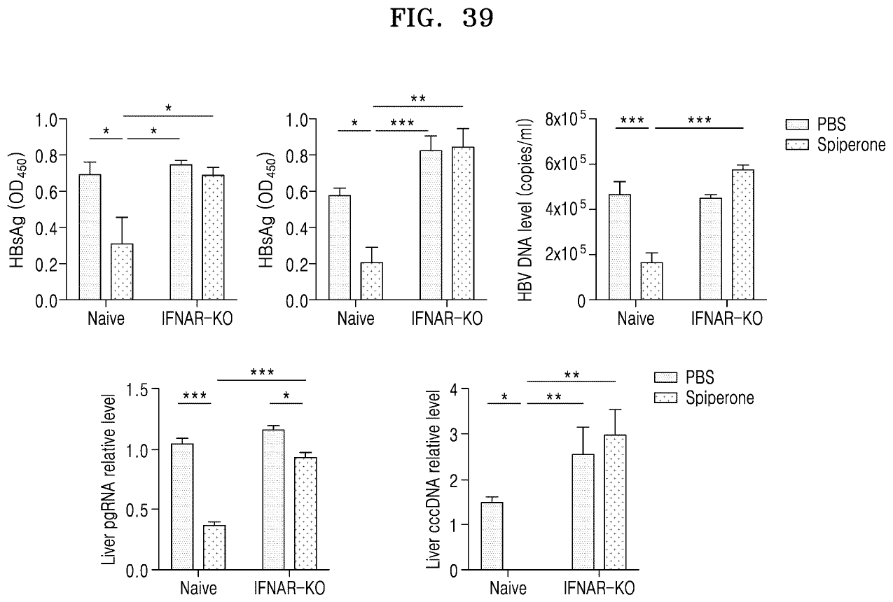

The experimental results thereof are shown in FIGS. 39 to 41.

FIG. 39 illustrates graphs showing the results of evaluating the levels of HBsAg, HBeAg, HBV DNA, liver pgRNA AND liver cccDNA to evaluate the dependence of spiperone on the type I IFN pathway by using IFNAR knockout mice.

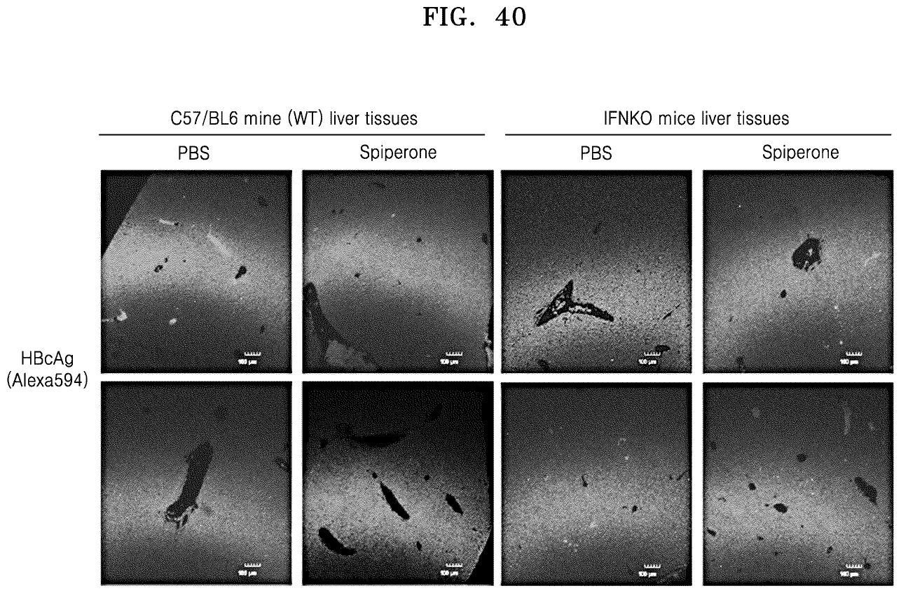

FIG. 40 illustrates the results of fluorescent immunostaining analysis to evaluate the dependence of spiperone on the type I IFN pathway by using IFNAR knockout mice.

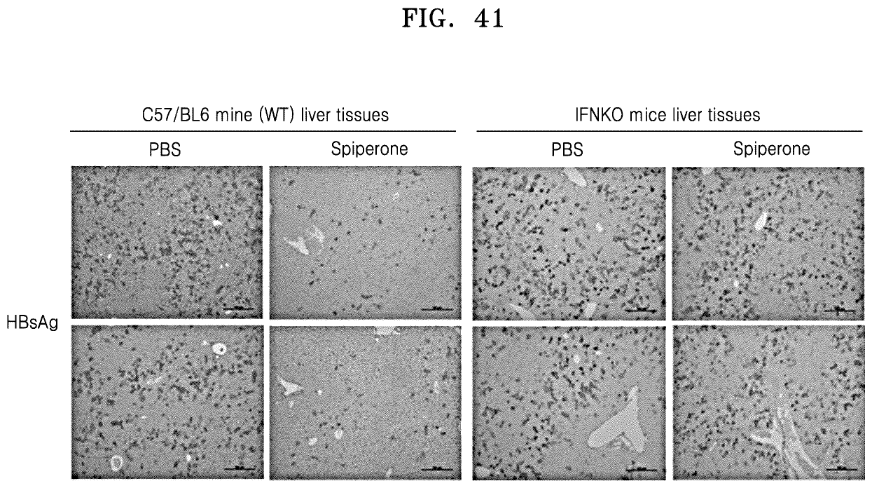

FIG. 41 illustrates the results of IHC staining analysis to evaluate the dependence of spiperone on the type I IFN pathway by using IFNAR knockout mice.

As illustrated in FIGS. 39 to 41, the levels of HBsAg, HBeAg, HBV DNA, pgRNA and cccDNA were all significantly reduced upon treatment with spiperone in wild-type (WT) mice, but most of these inhibitory effects were lost in IFNAR KO mice. In addition, in the immunofluorescence staining analysis of liver tissue, the fluorescence signal of HBcAg was significantly reduced in WT mice upon treatment with spiperone, whereas there was no significant difference in IFNAR KO mice compared to the PBS-treated group. Immunohistochemical staining (IHC) results for HBsAg also showed the same trend.

These results demonstrate in vivo that the antiviral effect of spiperone is dependent on the type I IFN signaling pathway.

### Example 6. Confirmation of Ability of Spiperone to Regulate and Eliminate HBV cccDNA

6.1 Confirmation of Epigenetic cccDNA Regulation Ability of Spiperone Through ChIP Analysis

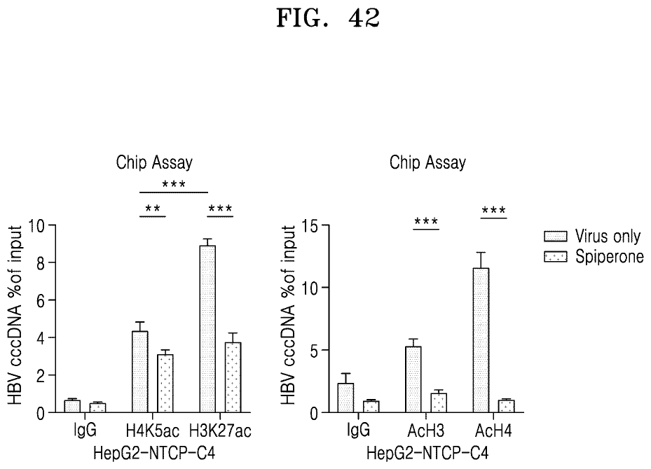

To confirm the effect of spiperone on the regulation of transcriptional activity of HBV cccDNA, chromatin immunoprecipitation (ChIP) assay was performed using HepG2-NTCP-C4 cells infected with HBV. Cells were infected with HBV virion, followed by treatment with spiperone, and histone proteins bound to cccDNA were immunoprecipitated using antibodies (H3K5ac, H3K27ac, AcH3, AcH4) against histone acetylation markers. Then, the binding levels of the histone markers were quantified through qPCR analysis using cccDNA-specific primers. The results thereof are shown in FIG. 42.

FIG. 42 illustrates the results of evaluating the epigenetic inhibitory mechanism by which spiperone acts on HBV cccDNA, by ChIP assay.

As illustrated in FIG. 42, it was confirmed that the levels of the histone acetylation markers (H3K5ac, H3K27ac, AcH3, AcH4) bound to HBV cccDNA were generally reduced in the spiperone-treated groups compared to the virus-only group. These results suggest that spiperone may induce antiviral effects through an epigenetic regulatory mechanism that suppresses transcriptional activity of HBV genes by inhibiting histone acetylation of HBV cccDNA.

6.2 Confirmation of cccDNA Inhibitory Ability of Spiperone Using AAV-HBV-Induced cccDNA Mouse Model (In Vivo)

Mice transduced with AAV-HBV1.04x (cccDNA mouse model) are an experimental animal model for evaluating HBV replication and cccDNA persistence. Unlike transgenic mice, this model features that the HBV genome is not integrated into host DNA, and allows the formation and regulation of HBV cccDNA within hepatocytes. By injecting spiperone into this mouse model, and then analyzing changes in various viral markers and cccDNA levels in liver tissues, the antiviral efficacy and cccDNA inhibitory ability of the corresponding drug may be evaluated.

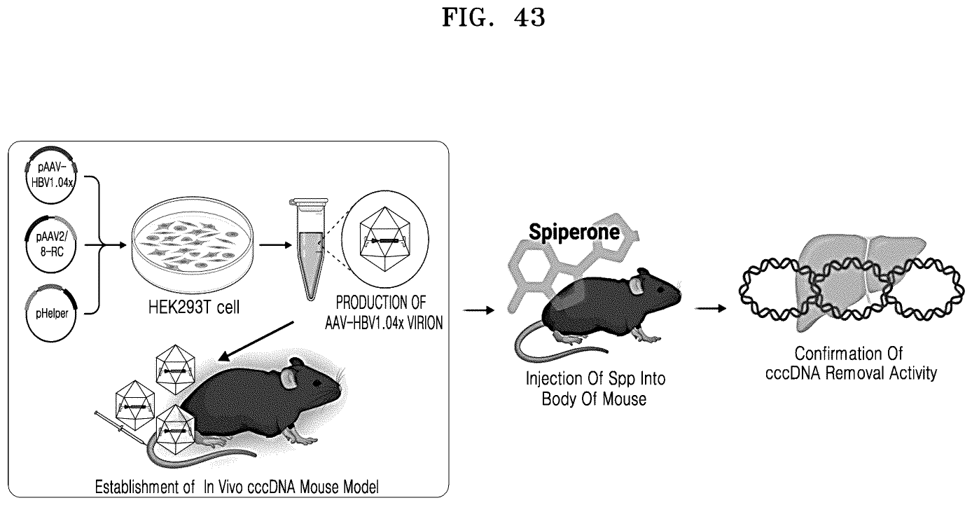

FIG. 43 is a schematic view of evaluating the ability of spiperone to reduce HBV cccDNA in an AAV-HBV mouse model (in vivo).

Specifically, to evaluate the in vivo cccDNA inhibitory ability of spiperone, an HBV infection mouse model was established by intravenously administering AAV-HBV1.04x (8×10¹⁰ vg/mouse) to C57BL/6 male mice, and then spiperone (1.5 mg/kg) or PBS was injected intraperitoneally (ip) daily for 4 weeks starting 7 days after infection. At the end of the experiment, serum and liver tissues were collected, and the levels of HBsAg, HBeAg, HBV DNA, pgRNA, and cccDNA were quantitatively analyzed using ELISA, RT-qPCR, and cccDNA-selective PCR, respectively. In addition, the expression of HBV antigens in liver tissue was evaluated through immunofluorescence staining for HBsAg and immunohistochemical staining for HBcAg.

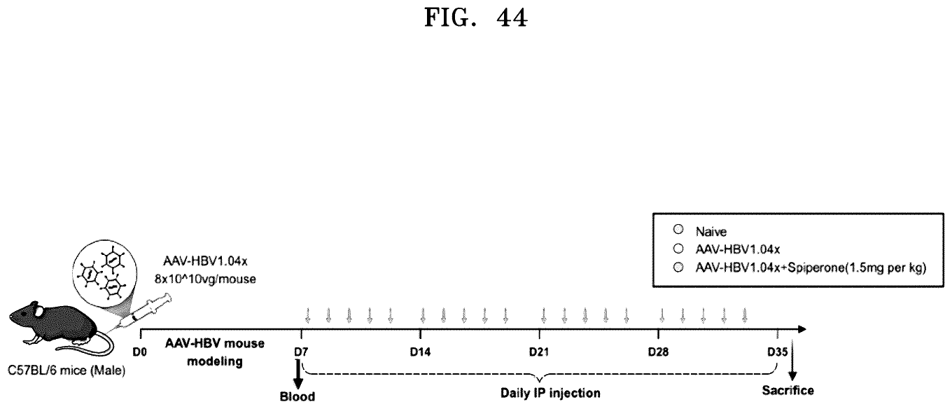

FIG. 44 illustrates an in vivo experimental schedule for evaluating the ability of spiperone to inhibit cccDNA.

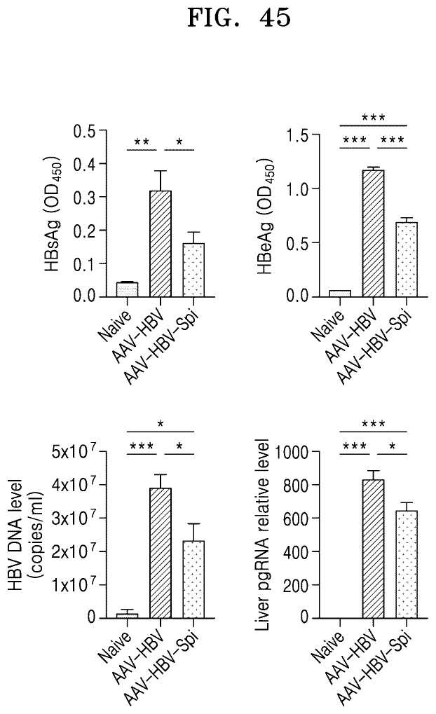

The experimental results thereof are shown in FIGS. 45 to 50.

FIG. 45 illustrates graphs showing the results of evaluating the levels of HBsAg, HBeAg, HBV DNA, and pgRNA to evaluate the anti-HBV activity of spiperone in an in vivo cccDNA mouse model.

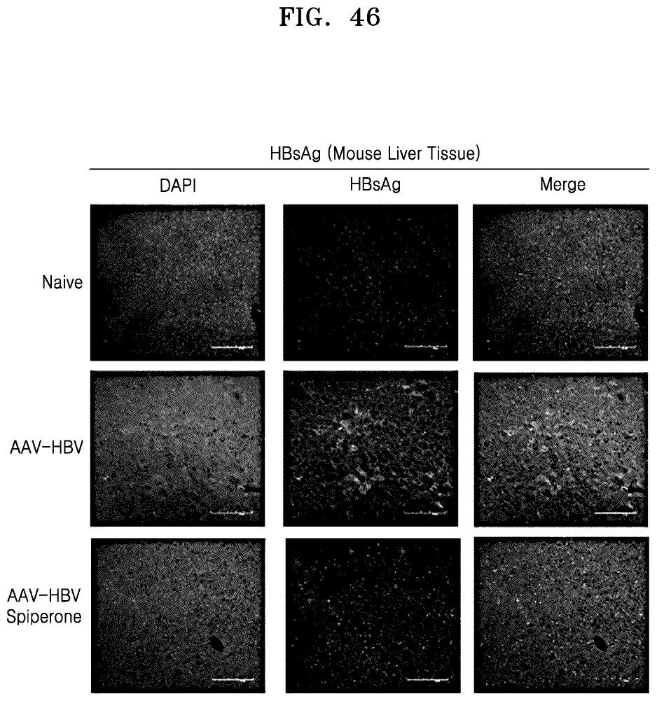

FIG. 46 illustrates the results of fluorescence immunostaining analysis to evaluate the anti-HBV activity of spiperone in an in vivo cccDNA mouse model.

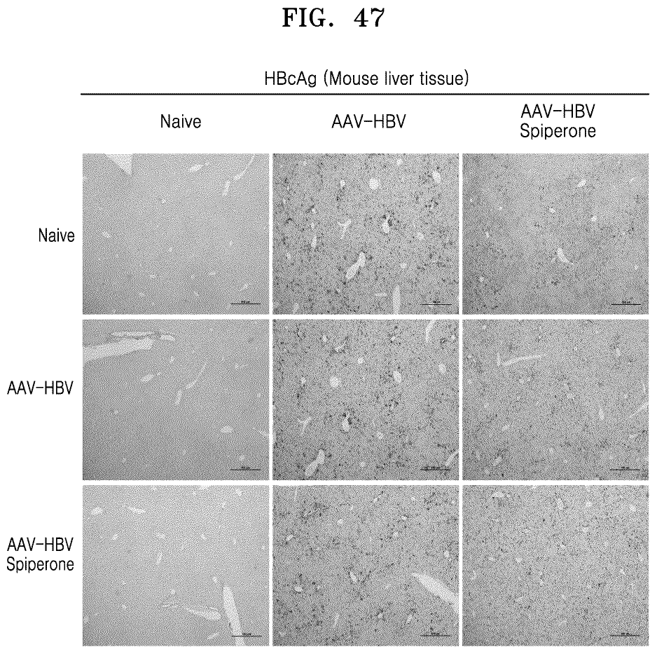

FIG. 47 illustrates the results of IHC staining analysis to evaluate the anti-HBV activity of spiperone in an in vivo cccDNA mouse model.

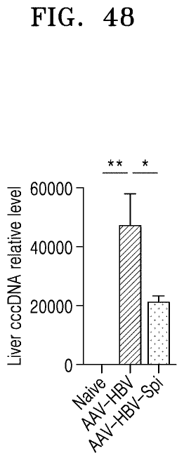

FIG. 48 illustrates the results of RT-qPCR quantitative analysis to evaluate the cccDNA inhibitory effect of spiperone by using an in vivo cccDNA mouse model.

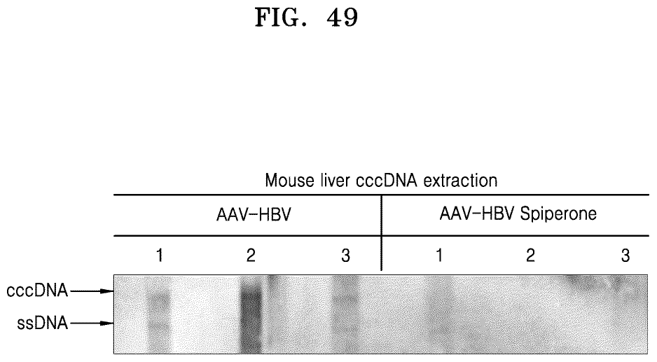

FIG. 49 illustrates the results of Southern blot analysis to evaluate the cccDNA inhibitory effect of spiperone by using an in vivo cccDNA mouse model.

FIG. 50 illustrates the results of cccDNA-selective PCR electrophoresis to evaluate the cccDNA inhibitory effect of spiperone by using an in vivo cccDNA mouse model.

As illustrated in FIGS. 45 to 47, the levels of HBsAg, HBeAg, and HBV DNA in the serum were significantly reduced in AAV-HBV-infected mice administered with spiperone, and the levels of pgRNA in liver tissues were also significantly inhibited through RT-qPCR analysis. The immunofluorescence staining (HBsAg) and immunohistochemical staining (HBcAg) of liver tissues also showed a remarkable reduction in HBV antigen expression in the spiperone-treated groups.

In particular, as illustrated in FIG. 48, as a result of quantifying cccDNA in liver tissues by RT-qPCR, a significant reduction in cccDNA levels was confirmed in the spiperone-treated groups, suggesting that spiperone can quantitatively suppress the presence of HBV cccDNA in vivo.

In addition, as illustrated in FIG. 49, Southern blot analysis further corroborated that cccDNA was reduced while single-stranded DNA species were unaffected. And, as illustrated in FIG. 50, in separate PCR electrophoresis analysis using cccDNA-selective and non-selective primers, strong cccDNA bands were detected only in the virus-only condition, whereas the intensity of these bands was significantly decreased in the spiperone-treated groups. This serves as qualitative evidence that spiperone substantially reduces cccDNA levels in liver tissues.

From the foregoing description, it will be understood by those of ordinary skill in the art to which the present disclosure pertains that the present disclosure may be embodied in other particular forms without changing the technical spirit or essential characteristics of the present disclosure. Therefore, the above-described experimental examples and examples should be construed as being provided for illustrative purposes only and not for purposes of limitation. The scope of the present disclosure should be construed as including all modifications and modified forms derived from the meaning and scope of the appended claims and the concept of equivalents thereto rather than the detailed description.

The present invention is the result of two research projects according to the following project information.
[Research Project Unique Number] 2460001485
[Research Project Number] RS-2024-00456525
[Department name] Ministry of Health and Welfare
[Research Project Management Agency name] Korea Health Industry Development Institute
[Research Project name] Infectious Disease Prevention and Treatment Technology Development Project-Technology Development for Unsolved Treatment Challenges
[Research Project Title] Development of a hepatitis B virus (HBV) cccDNA inhibitor using an endoplasmic reticulum stress inducer based on drug repurposing research
[Research performing Organization] Seoul National University R&DB Foundation
[Research Period] Jul. 1, 2024 to Dec. 31, 2024
[Research Project Unique Number] 2710079365
[Research Project Number] RS-2025-00553721
[Department name] Ministry of Science and ICT, National Research Foundation of Korea
[Research Project Management Agency name] National Research Foundation of Korea
[Research Project name] Individual Basic Research (Ministry of Science and ICT) (R&D)
[Research Project Title] Study on the mechanisms of hepatocellular carcinoma development and treatment strategies in HBV genotype C (Identification of the relationship between increased HBx gene expression due to the stability of the envelope antigen in refractory HBV genotype C mutant infection and the development of hepatocellular carcinoma, and in-depth study of treatment strategies)
[Research performing Organization] Seoul National University R&DB Foundation
[Research Period] Mar. 1, 2025 to Fab. 28, 2026

We claim:

1. A method for inhibiting or removing a viral cccDNA expression in a nucleus of a host cell, comprising administering spiperone or a pharmaceutically acceptable salt thereof to a subject in need thereof.

2. The method of claim 1, wherein the spiperone has antiviral activity by activating the PERK-elF2α-ATF4 signaling pathway, which is one of the endoplasmic reticulum stress pathways, and inducing the secretion of type I interferon.

3. The method of claim 1, wherein the spiperone has antiviral activity by activating the STING-TBK1-IRF3/7 signaling pathway, and inducing the secretion of type I interferon.

4. A method for inhibiting or removing a virus that produces cccDNA in a nucleus of a host cell, comprising administering spiperone or a pharmaceutically acceptable salt thereof to a subject in need thereof.

5. The method of claim 4, wherein the spiperone has antiviral activity by activating the PERK-elF2α-ATF4 signaling pathway, which is one of the endoplasmic reticulum stress pathways, and inducing the secretion of type I interferon.

6. The method of claim 4, wherein the spiperone has antiviral activity by activating the STING-TBK1-IRF3/7 signaling pathway, and inducing the secretion of type I interferon.

7. The method of claim 4, further comprising administering an antiviral agent.

8. The method of claim 4, wherein the antiviral agent is one or more selected from the group consisting of tenofovir, entecavir, adefovir, sofosbuvir, cidofovir, acyclovir, famciclovir, valacyclovir, ganciclovir, amprenavir, abacavir, ansamycin, darunavir, delavirdine, efavirenz, etravirine, hypericin, indinavir, lamivudine, lobucavir, nelfinavir, nevirapine, novapren, ritonavir, saquinavir, stavudine, tipranavir, virazole, ribavirin, zalcitabine, zidovudine, maraviroc, raltegravir, elvitegravir, didanosine, emtricitabine, lopinavir, atazanavir, enfuvirtide, and clevudine.

9. A method of preventing or treating virus infection, wherein a virus that produces cccDNA in the nucleus of a host cell, comprising administering spiperone or a pharmaceutically acceptable salt thereof to a subject in need thereof.

10. The method of claim 8, wherein the spiperone has antiviral activity by activating the PERK-elF2α-ATF4 signaling pathway, which is one of the endoplasmic reticulum stress pathways, and inducing the secretion of type I interferon.

11. The method of claim 8, wherein the spiperone has antiviral activity by activating the STING-TBK1-IRF3/7 signaling pathway, and inducing the secretion of type I interferon.

12. The method of claim 8, further comprising administering an antiviral agent.

13. The method of claim 8, wherein the antiviral agent is one or more selected from the group consisting of tenofovir, entecavir, adefovir, sofosbuvir, cidofovir, acyclovir, famciclovir, valacyclovir, ganciclovir, amprenavir, abacavir, ansamycin, darunavir, delavirdine, efavirenz, etravirine, hypericin, indinavir, lamivudine, lobucavir, nelfinavir, nevirapine, novapren, ritonavir, saquinavir, stavudine, tipranavir, virazole, ribavirin, zalcitabine, zidovudine, maraviroc, raltegravir, elvitegravir, didanosine, emtricitabine, lopinavir, atazanavir, enfuvirtide, and clevudine.


SEQUENCE LISTING (6 sequences)

SEQ ID NO: 1 - DNA, 19 bp, synthetic construct
  source 1..19: mol_type = other DNA
ccgtgtgcacttcgcttca

SEQ ID NO: 2 - DNA, 19 bp, synthetic construct
  source 1..19: mol_type = other DNA
gcacagcttggaggcttga

SEQ ID NO: 3 - DNA, 18 bp, synthetic construct
  source 1..18: mol_type = other DNA
ggggcgcacctctcttta

SEQ ID NO: 4 - DNA, 17 bp, synthetic construct
  source 1..17: mol_type = other DNA
aggcacagcttggaggc

SEQ ID NO: 5 - DNA, 20 bp, synthetic construct
  source 1..20: mol_type = other DNA
cactctatggaaggcgggta

SEQ ID NO: 6 - DNA, 20 bp, synthetic construct
  source 1..20: mol_type = other DNA
tgctccagctcctaccttgt